(19) United States
(12) Patent Application Publication
(10) Pub. No.: US 2019/0247649 A1
(43) Pub. Date: Aug. 15, 2019

(54) NANO-DEVICES FOR SKIN AND MUCOSAL MACROMOLECULE DELIVERY AND DETECTION

(71) Applicant: UNIVERSITY OF NORTH TEXAS, Denton, TX (US)

(72) Inventors: Lingqian CHANG, Denton, TX (US); Yongcun HAO, Denton, TX (US); Ifana MAHBUB, Denton, TX (US); Donghui ZHU, Denton, TX (US); Honglong CHANG, Xian (CN)

(21) Appl. No.: 16/271,209

(22) Filed: Feb. 8, 2019

Related U.S. Application Data
(60) Provisional application No. 62/628,617, filed on Feb. 9, 2018.

(51) Int. Cl. A61N 1/32; G03F 7/32; A61N 1/05; B32B 7/12; B32B 17/06; B32B 27/28; B32B 37/12; B32B 37/18; B32B 38/10; C23C 14/20; C23C 14/35; H04B 5/00; H01Q 1/38

(52) U.S. Cl. CPC A61N 1/327; B32B 2457/00; A61N 1/0502; B32B 7/12; B32B 17/064; B32B 27/281; B32B 37/12; B32B 37/182; B32B 38/10; C23C 14/205; C23C 14/35; H04B 5/0081; H01Q 1/38; B32B 2255/10; B32B 2255/205; B32B 2310/0806; B32B 2311/04; B32B 2379/08; B32B 2535/00; G03F 7/325

(57) ABSTRACT

In alternative embodiments, provided are products of manufacture and kits, and methods, for delivering macromolecules, including nucleic acids such as DNA and RNA, including genes and protein-encoding nucleic acids, to the skin, or the dermis or epidermis, and mucosa. In alternative embodiments, provided are products of manufacture and kits, and methods, for detecting macromolecules, including nucleic acids such as DNA and RNA, including genes and protein-encoding nucleic acids, in skin, epidermal or mucosal cells. In alternative embodiments, exemplary products of manufacture are physically flexible nanodelivery devices that are wearable, e.g., they can be worn as patches on the skin or mucosa. In alternative embodiments, nanodelivery devices provided herein are fabricated in a microelectrode—microfluidic—nanochannel configuration which can precisely deliver cargo into the ‘touched’ cells upon localized and safe-voltage electroporation. The on-skin electroporation can be wirelessly powered and controlled via on-chip near field communication (NFC) module. An accessory skin sensor can be simultaneously implemented on the same chip for skin impedance detection at the same time.

### TECHNICAL FIELD

This invention generally relates to medical devices and macromolecular delivery systems. In alternative embodiments, provided are products of manufacture and kits, and methods, for delivering macromolecules, including nucleic acids such as DNA and RNA, including genes and protein-encoding nucleic acids, to the skin, or the dermis or epidermis, and mucosa. In alternative embodiments, provided are products of manufacture and kits, and methods, for detecting macromolecules, including nucleic acids such as DNA and RNA, including genes and protein-encoding nucleic acids, in skin, epidermal or mucosal cells. In alternative embodiments, exemplary products of manufacture are physically flexible nanodelivery devices that are wearable, e.g., they can be worn as patches on the skin or mucosa. In alternative embodiments, nanodelivery devices provided herein are fabricated in a microelectrode—microfluidic—nanochannel configuration which can precisely deliver cargo into the ‘touched’ cells upon localized and safe-voltage electroporation. The on-skin electroporation can be wirelessly powered and controlled via on-chip near field communication (NFC) module. An accessory skin sensor can be simultaneously implemented on the same chip for skin impedance detection at the same time.

### BACKGROUND

Recently, a nanotransfection (TNT) chip (or NTC) incorporating nanochannels was developed for direct delivery of cargo such as genes, drug, etc., to the first layer of a tissue, e.g., for the skin, direct delivery to epidermis. This NTC was used to efficiently inject intracellularly to epithelial cells genetic reprogramming factors that forced conversion of these cells into endothelial cells. This NTC was also able to generate a therapeutic level of gene reprogramming for facilitating local wound healing. However, because this NTC is manufactured on silicon, it is brittle, rigid and expensive, which significantly limits its clinical application. Additionally, it is difficult to create nanochannel arrays on silicon NTC devices, which decreased its efficiency and increased the cost of its fabrication. Moreover, large cargo, such as plasmids, easily clog the silicon nanochannel, further hindering NTC operation and reproducibility of results.

### SUMMARY

In alternative embodiments, provided are products of manufacture for transferring macromolecules into a skin (e.g., a skin cell) or a mucosa (e.g., a mucosal cell), wherein the products of manufacture comprise a polyimide or equivalent, and the products of manufacture having embedded thereon or therein:

(a) a plurality of micro-channels forming a payload delivery zone, wherein each of the plurality of micro-channels is capable of holding or storing an aqueous solution,

wherein the each of the plurality of micro-channels extend to the surface, or can protrude from (or almost or substantially extend to) the surface, of the product of manufacture such that when all or a section of the product of manufacture is placed on the skin or the mucosa some, each of or substantially most of the plurality of micro-channels on the section of the product of manufacture makes complete, substantial or near contact with the skin or mucosa;

(b) an ultra-thin magnetic spiral antenna, or equivalent, and a near-field communication (NFC) chip, or equivalent, into or onto a section of the product of manufacture,

wherein the ultra-thin magnetic spiral antenna is operatively connected to the near-field communication (NFC) chip, wherein the ultra-thin magnetic spiral antenna can receive a remote electromagnetic signal and transmit the electromagnetic signal to the NFC chip, and the NFC chip is operatively connected to the plurality of micro-channels to further transmit the electromagnetic signal, or to generate a new or different signal, resulting in the plurality of micro-channels to discharge at least some (e.g., at least about 1%, 5%, 10%, 20%, 25% or more) or substantially all (e.g., at least about 80%, 85%, 90%, 95%, 98% or more) of their aqueous contents out of the plurality of micro-channels when the signal is transmitted; and

(c) a needle or nano-spike electrode capable of being inserted/placed in/on or in contact with the skin or mucosa, wherein the needle on one end is directly or indirectly connected (and is operatively connected) to the NFC chip, the NFC chip operatively connected to a micro-electrode under the micro-channels; and the needle or nano-spike electrode acts as an electrode connection to the skin or mucosa acting as a “bottom electrode”, and the NFC chip and micro-electrode under the micro-channels act as a “top” electrode capable of forming an electrode connection to the skin or mucosa when the plurality of micro-channels are in contact with (or nearly or substantially make contact with) or are placed on the skin or mucosal tissue, and the needle or nano-spike electrode when inserted into or in contact with (e.g., in substantial contact with, or at least in sufficient contact to transmit the signal) the skin or mucosa acts as the corresponding bottom electrode to complete the circuit,

wherein an electric field is transmitted onto or into the surface of the skin or mucosa each microchannel in contact with the skin or mucosa creates a small opening (optionally less than about 1 micron) on or through a cell's membrane, thereby electrophoretically driving a cargo from within the micro-channels (optionally macromolecules such as small molecules or nucleic acids) into the cell, thereby electroporating the cargo into the cell.

In alternative embodiments of the products of manufacture as provided herein, the polyimide or equivalent comprises or is a thermoplastic polyimide, or comprises or is a polyimide comprising: APICAL™; an a poly-oxydiphenylene-pyromellitimide, or KAPTON™; a biphenyl tetracarboxylic dianhydride (BPDA) polymer, or UPILEX™; VTEC PI™; NORTON TH™; KAPTREX™; or any combination thereof.

In alternative embodiments, the products of manufacture further comprise a Skin Sensor (SS) capable of measuring the connectivity between the product of manufacture and the skin or mucosa, wherein data generated by the SS is transmitted back to a receiving device (optionally a computer or a cell phone or equivalent comprising a corresponding receiving and transmitting device).

In alternative embodiments, provided are methods for making a product of manufacture as described herein, comprising:

(a) coating a substrate comprising a polymeric organosilicon compound, optionally a silicone, a polydimethylsiloxane (PDMS) or an equivalent, on a glass or equivalent, wherein the substrate as an adhesive layer for a polyimide or equivalent;

(b) bonding the polyimide or equivalent to the polymeric organosilicon compound or equivalent using a vacuum followed by a heat treatment,

wherein optionally the vacuum is below about 1 kPa, the temperature is between about 60° C. to 80° C., and/or the bonding time is between about 1 hour (h) to about 3 h;

(c) sputtering chromium and gold on the surface of the photoresist and polyimide or equivalent (a Cr/Au sputtered layer);

wherein optionally the chromium and gold is sputtered on the surface of the photoresist and polyimide or equivalent using magnetron sputtering equipment, and/or the Cr layer is between about 20 nm to 50 nm, and/or the Au layer is between about 200 nm to 500 nm;

(d) patterning a positive photoresist composition on the polyimide or equivalent as a sacrifice layer,

wherein optionally the positive photoresist comprises EPI 680™ (Everlight Chemical, Taiwan), the thickness of patterned photoresist is between about 2 μm to 4 μm, and/or the pattern shape is determined by the Cr/Au layer in step (c), or the pattern has a complementary relationship with Cr/Au structure;

(e) lifting the photoresist, optionally by soaking in an acetone or equivalent solution, optionally for between about 10 min to about 30 min to remove the photoresist completely;

(f) patterning a plurality of micro-channels on the product of manufacture to form a payload delivery zone,

wherein optionally a positive reflowable photoresist AZ P4620™ (Microchemicals GmbH, Ulm, Germany) was used to fabricate a master microchannel mold (optionally as described in Huang, et al. Biomed Microdevices (2012) 14: 873),

wherein the each of the plurality of micro-channels extend to (or protrude from, or almost or substantially extend to) the surface of the product of manufacture such that when all or a section of the product of manufacture is placed on the skin or mucosa each of the plurality of micro-channels on the section of the product of manufacture on the skin makes contact (or nearly or substantially makes contact with) with the skin;

(g) applying an ultra-thin magnetic spiral antenna and a near-field communication (NFC) chip into or onto a section of the product of manufacture, wherein the ultra-thin magnetic spiral antenna is operatively connected to the near-field communication (NFC) chip, wherein the ultra-thin magnetic spiral antenna can receive a remote electromagnetic signal and transmit the signal to a near-field communication (NFC) chip, and the NFC chip is operatively connected to the plurality of micro-channels to further transmit the signal and result in the plurality of micro-channels discharging their aqueous contents out of the plurality of micro-channels when the signal is transmitted;

(g) stripping the polyimide from the glass, wherein optionally the polyimide can be stripped directly by hand.

In alternative embodiments of the methods, the polyimide or equivalent comprises or is a thermoplastic polyimide, or comprises or is a polyimide comprising: APICAL™; an a poly-oxydiphenylene-pyromellitimide, or KAPTON™; a biphenyl tetracarboxylic dianhydride (BPDA) polymer, or UPILEX™; VTEC PI™; NORTON TH™; KAPTREX™; or any combination thereof.

In alternative embodiments of the methods or the products of manufacture, the plurality of micro-channels each comprise an aqueous solution, and optionally the aqueous solution comprises a payload, and optionally the payload comprises a macromolecule or a small molecule, and optionally the macromolecule comprises a nucleic acid, and optionally the nucleic acid comprises a DNA or an RNA, optionally a gene or protein-encoding nucleic acid.

In alternative embodiments, the methods or the products of manufacture further comprise either: (a) directly affixing or attaching a needle or a nano-spike electrode (optionally a gold needle or nano-spike electrode, optionally having a diameter of between about 0.5 mm to about 1 mm) to the product of manufacture; or, (b) indirectly connecting a needle or a nano-spike electrode to the product of manufacture by a wire, wherein the needle or the nano-spike electrode is connected to an NFC chip by a wire bonding.

In alternative embodiments, provided are kits comprising: a product of manufacture as provided herein, or a product of manufacture made by a method as provided herein, wherein optionally the further comprises software for downloading or loading onto a phone, a computer or an equivalent device for allowing or enabling interaction between a user and the product of manufacture, and data transmission from the product of manufacture to the phone, computer or equivalent device, and presentation of the transmitted data to the user, wherein optionally the software is packaged as an app for the phone or equivalent device.

In alternative embodiments, provided are Uses of a product of manufacture as provided herein, or a kit as provided herein, for transferring macromolecules into a skin or a mucosal cell.

In alternative embodiments, provided are methods for delivering a payload to skin or mucosal cells, or to dermal or epidermal cells, comprising:

(a) applying a product of manufacture as provided herein to a skin or a mucosa or a skin or a mucosal cell, wherein the plurality of micro-channels of the product of manufacture make complete, substantial or near contact with the skin; and

(b) transmitting a sufficient electromagnetic signal to the product of manufacture for reception by the ultra-thin magnetic spiral antenna, which transmits this signal to the near-field communication (NFC) chip, and the NFC chip, which is operatively connected to the plurality of micro-channels, further transmits the signal to result in the plurality of micro-channels to discharge some or substantially all or all of their aqueous contents (the payload) out of the plurality of micro-channels,

and optionally the electromagnetic signal is also sufficient to result in an electroporation of some or substantially all or all of the payload into the skin or mucosal cells.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A illustrates images of an exemplary product of manufacture (e.g., a device) as provided herein: left panel, showing the flexibility of an exemplary nanodevice which in one embodiment comprises a polyimide substrate; middle panel, illustrating components in the nanodevice; and, right panel, showing a flexible device as placed in close contact upon the skin.

FIG. 1B schematically illustrates an exemplary product of manufacture as provided herein, including placement and orientation of a skin sensor, microelectrode, microfluidic section, skin electroporation section, and on-chip NFC module.

FIG. 2A-H illustrates an exemplary fabrication method for an exemplary product of manufacture (e.g., a nanodevice) as provided herein, including:

FIG. 2A schematically illustrates coating a polymeric organosilicon compound, e.g., a silicone such as a polydimethylsiloxane (PDMS), or equivalent, on a glass or equivalent, wherein the PDMS), or equivalent acts as an adhesive layer for a polyimide or equivalent;

FIG. 2B schematically illustrates bonding the polyimide or equivalent (the polyimide or equivalent including a thermoplastic polyimide, or, e.g., APICAL™; a poly-oxydiphenylene-pyromellitimide, or KAPTON™; a biphenyl tetracarboxylic dianhydride (BPDA) polymer, or UPILEX™; VTEC PI™; NORTON TH™; and, KAPTREX™), to the polymeric organosilicon compound, e.g., a PDMS, with a vacuum, followed by a heat treatment;

FIG. 2C schematically illustrates patterning a photoresist composition on the polyimide or equivalent as the sacrifice layer;

FIG. 2D schematically illustrates sputtering chromium and gold on the surface of the photoresist and polyimide or equivalent (a Cr/Au sputtered layer);

FIG. 2E schematically illustrates lifting the photoresist;

FIG. 2F schematically illustrates patterning the micro-channels; and

FIG. 2G schematically illustrates stripping the polyimide from the glass (the polyimide stripped from the glass).

FIG. 2H schematically illustrates a key indicating specific elements of the device for FIG. 2A-G.

FIG. 2I schematically illustrates the skin sensor (SS), GD zone and WP zone on an exemplary device.

FIG. 3A-D illustrate various aspects of an exemplary device as provided herein:

FIG. 3A illustrates images of three panels showing increasingly magnified images of an exemplary Macromolecule (or Gene) Delivery zone (GD): Top Panel: devices can be batch-produced from one big polymer sheet; lower Left Panel: a zoom-in image of a micrograph shows the features of microchannel connection on a single device; Lower Right Panel: a further zoom-in image shows one square region of microchannel where the circle arrays are pillars supporting a top nanopore membrane and defining an electric field focusing on the nanochannel.

FIG. 3B schematically illustrates an exemplary product of manufacture (e.g., device) as provided herein, and illustrates how a 99% potential drop is distributed over a nanopore for macromolecular (e.g., gene) electrophoresis, as discussed in detail, below.

FIG. 3C schematically illustrates a cross-sectional schematic showing how a pulsed electrode field is applied between a top electrode (nano-spike electrode) and a bottom electrode (micro-electrode array) of an exemplary device patched on the skin, as discussed in detail, below.

FIG. 3D schematically illustrates two images of an exemplary mounting nanopore membrane (e.g., a polycarbonate track etched membrane) on a microchannel, the nanopore membrane forming an insulated microfluidic channel for flow in and flow out the macromolecule solutions.

FIG. 4A-C schematically illustrates an exemplary product of manufacture (e.g., device) as provided herein:

FIG. 4A schematically illustrates how a needle, or a so-called “nano-spike electrode” of an exemplary product of manufacture on one end is directly connected (and is operatively connected) to the NFC chip, where the NFC chip is operatively connected to a micro-electrode under the micro-channels, where the needle (or “nano-spike electrode”) acts as an electrode connection to the skin or mucosa.

FIG. 4B schematically illustrates an exemplary NFC system as provided herein that wirelessly powers the NEP device and optionally also has a separate data communication link, as discussed in detail, below.

FIG. 4C schematically illustrates an exemplary wireless telemetry system between the cell-phone and an exemplary nano-device patched in or on the skin.

FIG. 5 schematically illustrates an exemplary product of manufacture as provided herein showing the operative and spatial relationship of the antenna, wireless power transfer (WPT), drug/gene loading/electroporation/delivery zone and in-skin electrode, or microelectrode.

FIG. 6A-H schematically illustrate an exemplary fabrication method for an exemplary product of manufacture (e.g., a nanodevice) as provided herein, including:

FIG. 6A schematically illustrates coating polydimethylsiloxane (PDMS) on glass as an adhesive layer;

FIG. 6B schematically illustrates bonding a polyethylene naphthalate (PEN) to PDMS with vacuum and heat treatment;

FIG. 6C schematically illustrates patterning the photoresist on polyethylene naphthalate (PEN) as the sacrifice layer;

FIG. 6D schematically illustrates sputtering Au with the thickness of about 100 nm;

FIG. 6E schematically illustrates lifting the photoresist off using acetone;

FIG. 6F schematically illustrates patterning the micro-channels using AZ4620™ (a photoresist compound comprising 1-methoxy-2-propanol acetate, cresol novolak resin, diazonaphtoquinonesulfonic esters);

FIG. 6G schematically illustrates stripping the polyethylene naphthalate (PEN) from glass;

FIG. 6H schematically illustrates a key indicating specific elements of the device for FIG. 6A-G.

FIG. 7A-B each schematically illustrate an exemplary product of manufacture (e.g., a nanodevice) as provided herein, wherein the product of manufacture of FIG. 7A has an antenna with 9 turns, and an exemplary product of manufacture (e.g., a nanodevice) as provided herein, wherein the product of manufacture of FIG. 7A has an antenna with 6 turns.

FIG. 8A-B each schematically illustrate an exemplary product of manufacture (e.g., a nanodevice) as provided herein, wherein the (incompletely fabricated) product of manufacture of FIG. 8A illustrates an integrated device before microfluidic channel patterning, where the electrode and antenna are fully patterned; and the product of manufacture of FIG. 8B illustrates an integrated device with completed microfluidic channel patterning.

FIG. 9A-B each schematically illustrate an exemplary product of manufacture (e.g., a skin nanodevice) as provided herein, wherein FIG. 9A upper image illustrates a close-up image of a gene/drug loading zone of an exemplary device, and the lower image illustrates a close up image of the nucleic acid (e.g., gene)/drug loading zone, which is bonded with polycarbonate, and is nanoporous; and, FIG. 9B illustrates an exemplary product of manufacture (e.g., a skin nanodevice) as provided herein, and the lower image illustrates silicon micro-needles, which can have a height of e.g., about 200 μm (or can be anywhere from between about 50 and 500 μm), where the micro-needle can act as an “in-skin” electrode.

FIG. 10 schematically illustrates loading of nucleic acids (e.g., genes) into microfluidic channels of an exemplary product of manufacture, the so-called “wetting process” or procedure used in the manufacture of the device.

FIG. 11A-B schematically illustrate properties of electrodes and microfluidic channels of an exemplary product of manufacture (e.g., a nanodevice), wherein FIG. 11A illustrates the thickness of gold (Au), the top half of the illustrated device, and polyethylene naphthalate (PEN), the lower half of the illustrated device, where the thickness of the Au is measured by optical profiler; and FIG. 11B illustrates the thickness of the photoresist agent (e.g., AZ4620, or equivalent) and PEN in a cross-section of the device, as indicated by the line in the mid-section of the figure, noting that the thickness of the photoresist agent (also as measured by optical profiler) is 15 μm (or can be anywhere from between about 5 and 50 μm).

FIG. 12 schematically illustrates a block diagram of a wireless power transfer (WPT) module, or WPT system, of an exemplary product of manufacture, including a transmitting (TX) module as the left image and a receiving (RX) module as the right image.

FIG. 14A-B schematically illustrate an exemplary design and fabrication of an exemplary WPT module, or system, with FIG. 14A illustrating a copper clad FR4 substrate, and FIG. 14B illustrating a flexible polyethylene naphthalate (PEN) substrate.

FIG. 14A-B schematically illustrate an exemplary transmitter (TX) module; with FIG. 14A illustrating a TX on a copper clad FR4, and FIG. 14B illustrating a schematic of an exemplary TX inductor model, with a signal generator, as discussed in further detail, below.

FIG. 15A-B schematically illustrate an exemplary receiver (RX) module; with FIG. 15A illustrating the RX on a PEN substrate, and FIG. 15B illustrating a schematic of an exemplary RX inductor model, as discussed in further detail, below.

FIG. 16A-B illustrate design optimization and validation of an exemplary product of manufacture (e.g., a nanodevice), where FIG. 16A graphically illustrates a simulation result of inductance and Q-factor of RX coil (using a high frequency structure (HFSS) software), where inductance (nH) is a function of frequency (MHz); and, FIG. 16B schematically illustrates the TX and RX coils used in the simulation, where the Q factor for the TX coil is 37 and the inductance is 38 nH, and where for the RX coil the Q factor is 3 and inductance is 676 nH.

FIG. 16A-B illustrate design optimization and validation of an exemplary product of manufacture (e.g., a nanodevice), where FIG. 16A graphically illustrates a simulation result of inductance and Q-factor of RX coil, where inductance (nH) is a function of frequency (MHz); and, FIG. 16B schematically illustrates the TX and RX coils used in the simulation, where the Q factor for the TX coil is 37 and the inductance is 38 nH, and where for the RX coil the Q factor is 3 and inductance is 676 nH.

FIG. 17A-B illustrate design optimization and validation of an exemplary product of manufacture (e.g., a nanodevice), where FIG. 17A graphically illustrates a simulation result of return loss (S₁₁) of the TX and RX modules, where Return Loss (dB) is a function of frequency (MHz); and, FIG. 17B schematically illustrates the TX and RX coils used in the simulation, where the Q factor for the TX coil is −9.83 dB at 4.2 MHz, and the RX coil is −28.3 dB at 3.95 MHz.

FIG. 18 graphically illustrates design optimization and validation of an exemplary product of manufacture (e.g., a nanodevice), where the TX coil is above a 6-layer tissue module with 1.5 mm of air gap; and the transmitted power is (Pᵢₙ) is 15 dBm at 4 MHz; and the RX coil is above and below the skull layer; the simulated power transfer (PTE): above the skull later=PTE is 20%, and below the skull layer=PTE is 2.4%, where:

$$η = \frac{{P}_{out}}{{P}_{in}} \cdot 100 \% .$$

FIG. 19A-B illustrate a design optimization and validation of an exemplary product of manufacture (e.g., a nanodevice), where FIG. 19A schematically illustrates a high frequency structure (HFSS) SAR (or “Specific Absorption Rate”; it is the measure of the rate at which electromagnetic energy is absorbed by the tissue) setup with the RX coil below the skull and TX above the skull; and FIG. 19B graphically illustrates the SAR for the different tissue layer depths (from FIG. 19A).

FIG. 20 illustrates an image of an exemplary test set-up at a 10 mm distance, where transmitted power (Pᵢₙ) is 15 dBm at 4 MHz; the distance between the RX and TX coils is between about 5 mm and 10 mm; the signal generator is an EXG N5171B™ (Keysight Technologies, Santa Rosa, Calif.) analog signal generator; and the oscilloscope is an INFINIIVISION (infiniiVision) DSOX3014A™ oscilloscope (Keysight Technologies, Santa Rosa, Calif.).

FIG. 21 graphically illustrates measuring PTE through air, where measured RX output voltage is 230.2 mVₚₚ at 5 mm and 168.8 mVₚₚ at 10 mm; Power Received is −26 dBm at 5 mm and −28 dBm at 10 mm; Measured Power Transfer Efficiency (PTE) is 7.6% at 5 mm and 4.1% at 10 mm; Input impedance of Schottky Diode: R≈22 Ω is Forward Voltage: V_{f}=0.22 V and Forward Current: I_{f}=10 mA; and the graphic measures TX and RX voltages, where transmitter voltage (V) is a function of time in seconds (s):

$$\frac{{P}_{out}}{{P}_{in}} \times 100 \%$$

$${P}_{out} = 10 \cdot {log}_{10} ( \frac{{Vpp}_{RX}^{2}}{{R}_{in}} ) .$$

FIG. 22A-C illustrate the delivery of large size gene materials such as e.g., NF2-CRISPR-Cas9 plasmids (9 kilobase, kb) into melanoma cells for gene therapy based melanoma treatment: FIG. 21A schematically illustrates a CRISPR Cas9 mechanism for knocking out and downregulating the NF2 gene; FIG. 22B graphically illustrates a plot showing that the transfection efficiency of an exemplary skin nanodevice as provided herein provides a significantly higher efficiency than a commercial electroporation (BioRad), where transfection efficiency (%) for exemplary “skin patch” devices as provided herein, and for known commercial electroporation devices, are shown; and FIG. 22C illustrate images fluorescence images of the cells after gene transfection.

FIG. 23A-B illustrate a comparison of delivery methods for dacarbazine into A375 cell, and illustrates fluorescence images and plots illustrating that an exemplary skin nanodevice as provided herein is better for chemo drug delivery induced melanoma treatment than known methods: FIG. 23A illustrates fluorescence images after treating cells with dacarbazine (after transfecting cells with dacarbazine using NEP 15V); FIG. 23B graphically illustrates cell viability after direct treatment of cells with dacarbazine, where the plot shows that an exemplary skin nanodevice as provided herein has a higher efficiency for melanoma cell inhibition as compared to other methods, including direct drug treatment and chemical methods using commercial lipofectamine 2000.

FIG. 24A-B graphically illustrate plots showing dosage controllability for chemotherapeutic drug delivery using. Dacarbazine (FIG. 24A) and cisplatin (FIG. 24B).

Like reference symbols in the various drawings indicate like elements.

### DETAILED DESCRIPTION

In alternative embodiments, provided are products of manufacture and kits, and methods, for delivering macromolecules, including nucleic acids such as DNA and RNA, including genes and protein-encoding nucleic acids, to the skin or epidermis and mucosa. In alternative embodiments, provided are products of manufacture and kits, and methods, for detecting macromolecules, including nucleic acids such as DNA and RNA, including genes and protein-encoding nucleic acids, in skin, epidermal or mucosal cells.

In alternative embodiments, provided is a flexible nanoscale macromolecule (e.g., including nucleic acids such as DNA and RNA, including genes and protein-encoding nucleic acids) delivery device for skin patching, epidermal and dermal gene delivery, the device comprising both wireless communication and a powering abilities.

In alternative embodiments, exemplary devices described herein solve many or all problems associated with known nanotransfection chips (or NTCs), including e.g., brittle, rigid and expensive silicon-based NTCs, which significantly limit clinical applications; and, NTC nanochannel operating difficulties (e.g., large cargo, such as plasmids, easily clog the nanochannel, further hindering nanotransfection chip operation and reproducibility of results), which decrease NTC efficiency and increase the cost of NTC fabrication.

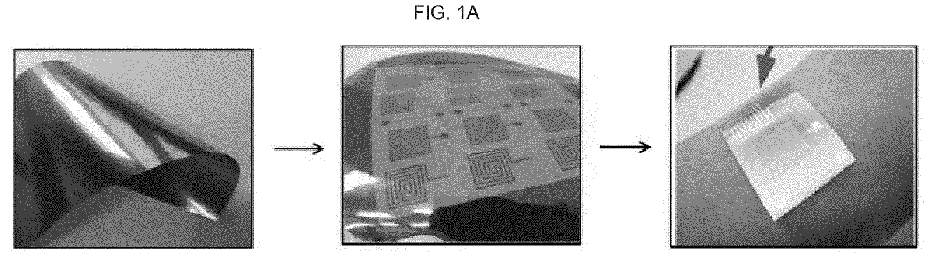

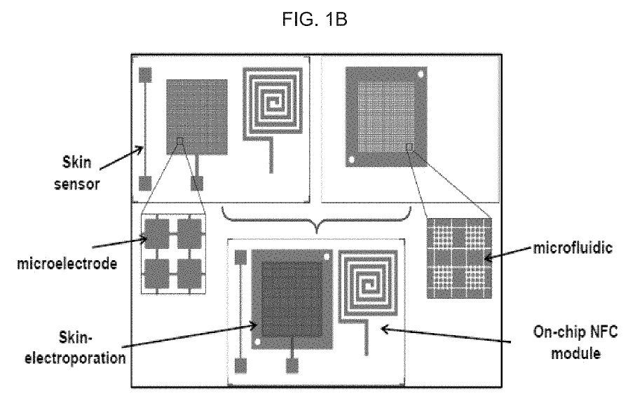

For example, in alternative embodiments, exemplary devices described herein are flexible nanodevices which can bend and patch on the skin or mucosa, and have a microelectrode—microfluidic—nanochannel configuration that can precisely deliver cargo into the ‘touched’ cells upon localized and safe-voltage electroporation, as illustrated e.g., in FIG. 1A, left panel, showing the flexibility of an exemplary nanodevice, which in one embodiment comprises a polyimide substrate; FIG. 1A, middle panel, illustrating components in the nanodevice; and, FIG. 1A, right panel, showing a flexible device placed upon the skin, e.g., as a skin patch or overlay for, e.g., remote electronically controlled macromolecule delivery directly to the upper layer of the skin.

In alternative embodiments, the on-skin electroporation is wirelessly powered and controlled via an on-chip near field communication (NFC) module. In alternative embodiments, an accessory skin sensor is simultaneously implemented on the same chip for skin impedance detection at the same time as the wireless on-skin electroporation. For example, FIG. 1B illustrates an exemplary nanodevice as provided herein comprising a skin sensor, a microelectrode, a skin electroporation, a microfluidic, and an on-chip near-field communication (NFC) module and an ultra-thin magnetic spiral antenna.

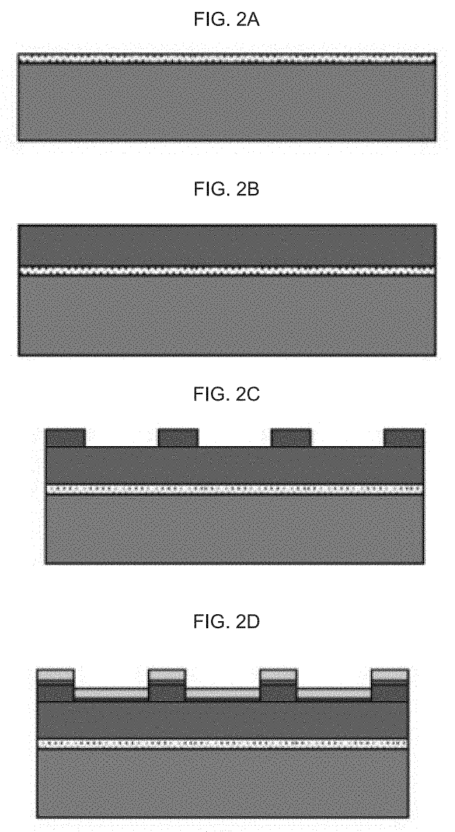

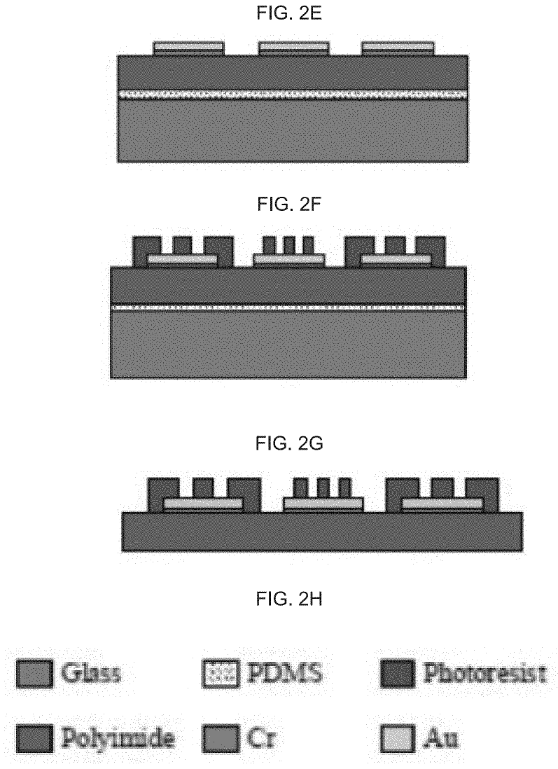

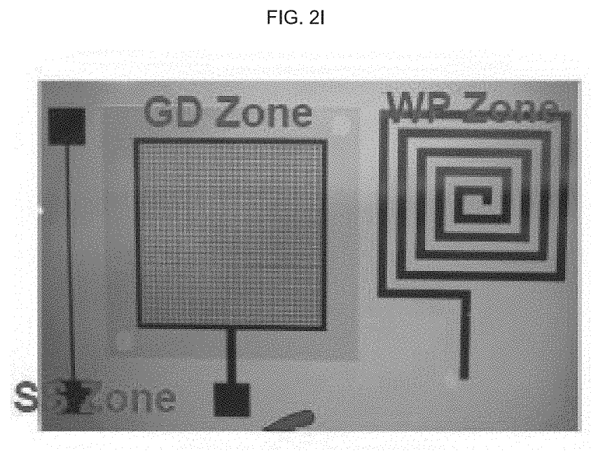

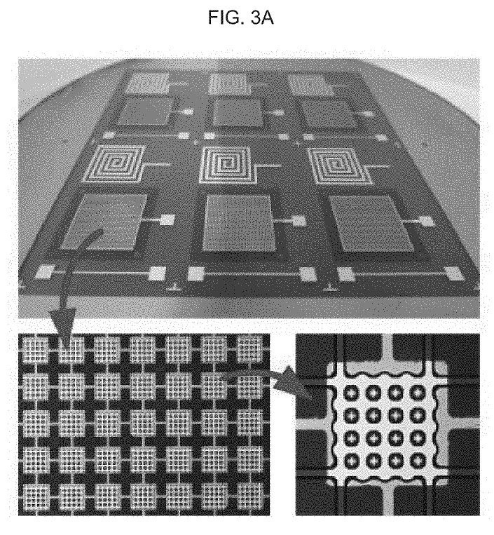

As exemplary fabrication method for a nanodevice as provided herein is illustrated in FIG. 2A-F.

In alternative embodiments, at the device level, a nanoscale platform is manufactured on the polymeric substrate (e.g., a polyimide or equivalent) so that the entire platform can be easily bended and adjusted for different skin or mucosal shapes.

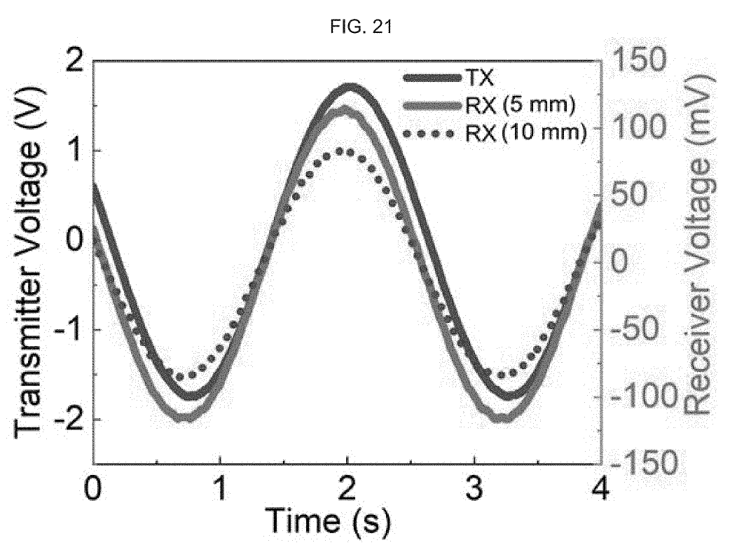

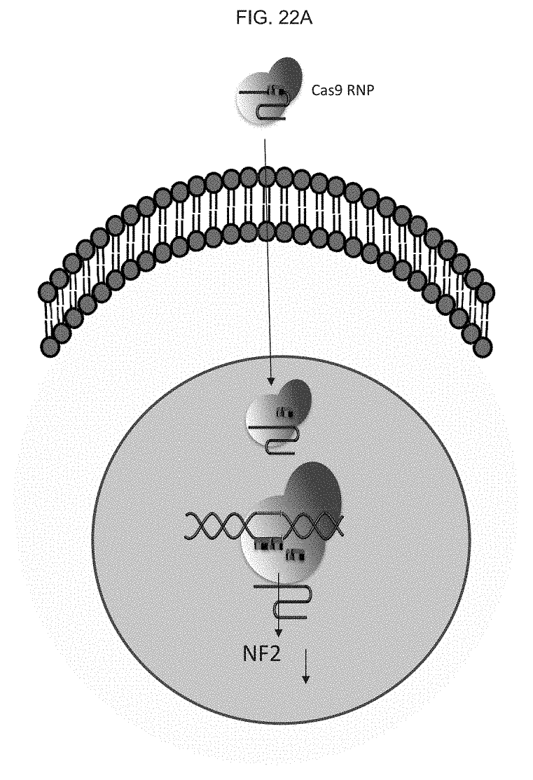

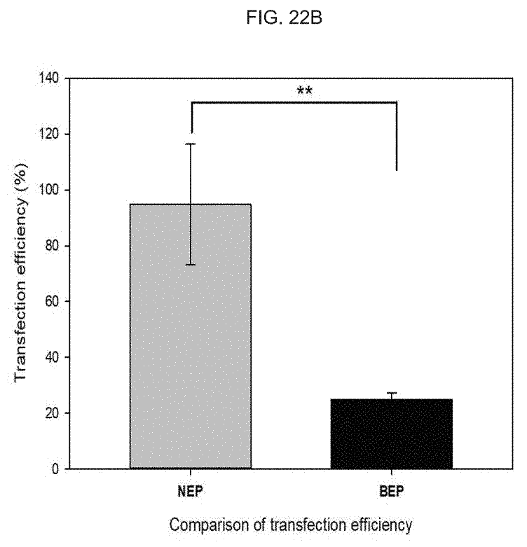

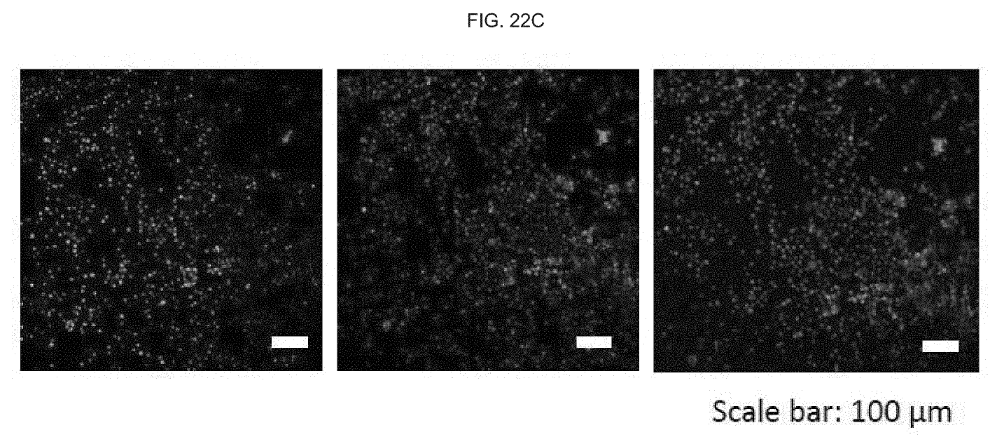

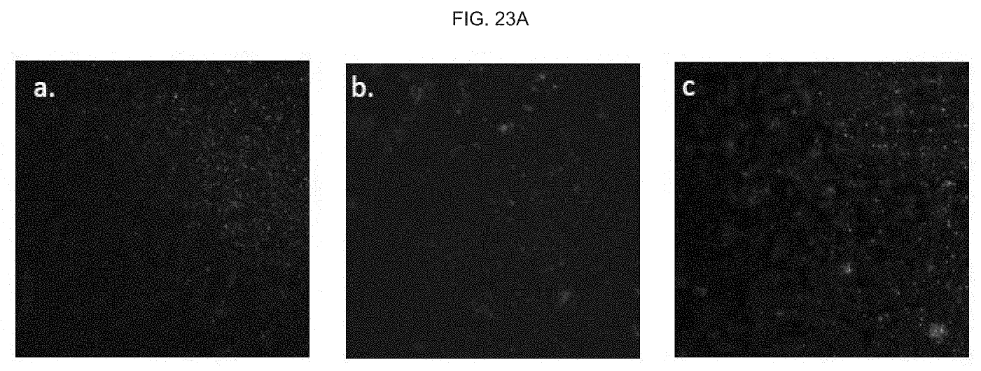

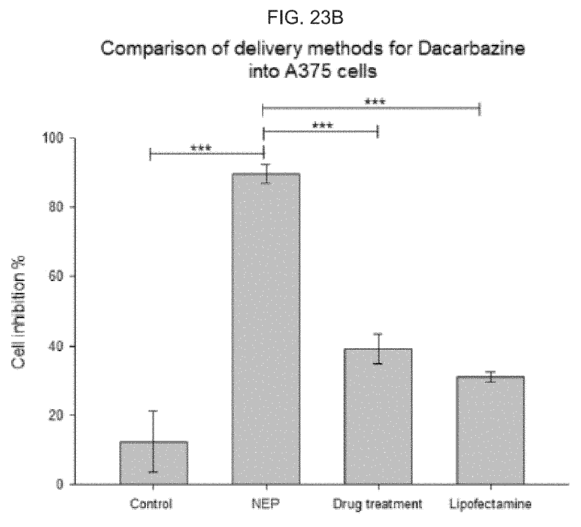

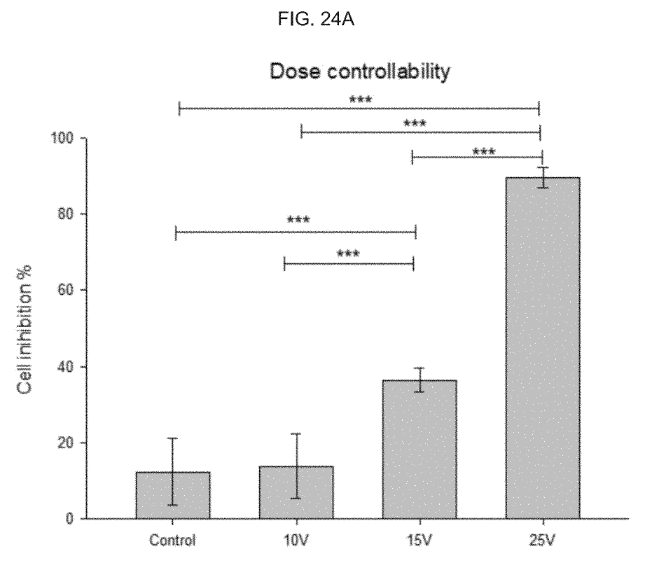

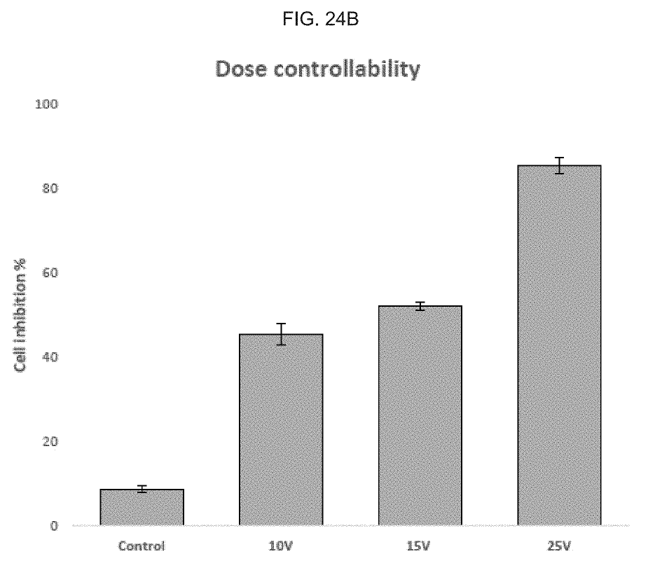

In alternative embodiments, the device comprises three functional zones, including: (1) a Macromolecule (or Gene) Delivery zone (GD); (2) a Wireless Power zone (WP); and, (3) a Skin Sensor (SS). The three zones can be simultaneously fabricated on a polymer substrate using, e.g., cleanroom technology. FIG. 21 illustrates the skin sensor (SS), GD zone and WP zone on an exemplary device.

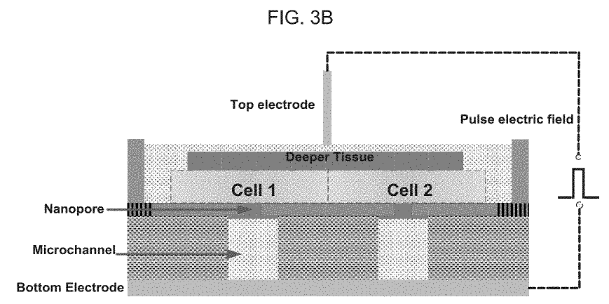

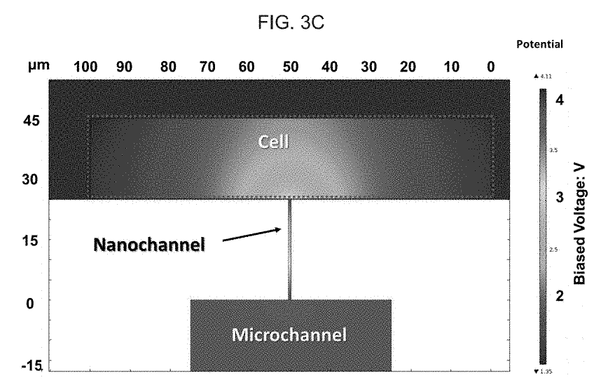

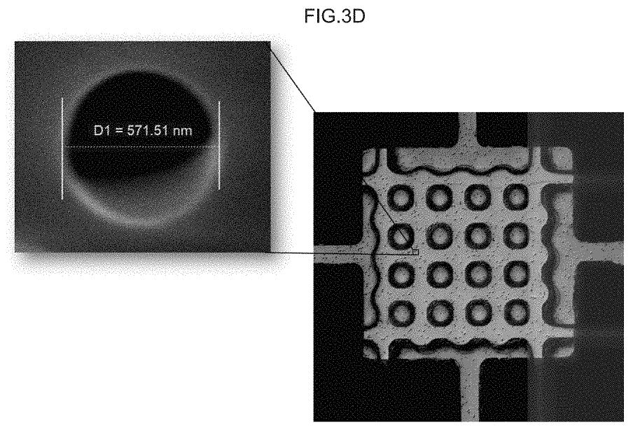

In alternative embodiments, in the GD zone, a bottom micro-electrode array (which can have a dimension of about 50 microns, see FIG. 3C) and a microfluidic channel array (which can have a dimension of about 50 microns, see FIG. 3C) are sequentially patterned on the polymeric substrate by deposition of a thin layer of gold and photolithography. Nanopores can be constructed on the microfluidic channel array via thermal lamination of polyimide or equivalent nano-porous membrane (which can have a diameter of about 0.6 microns, and/or a thickness of about 10 microns).

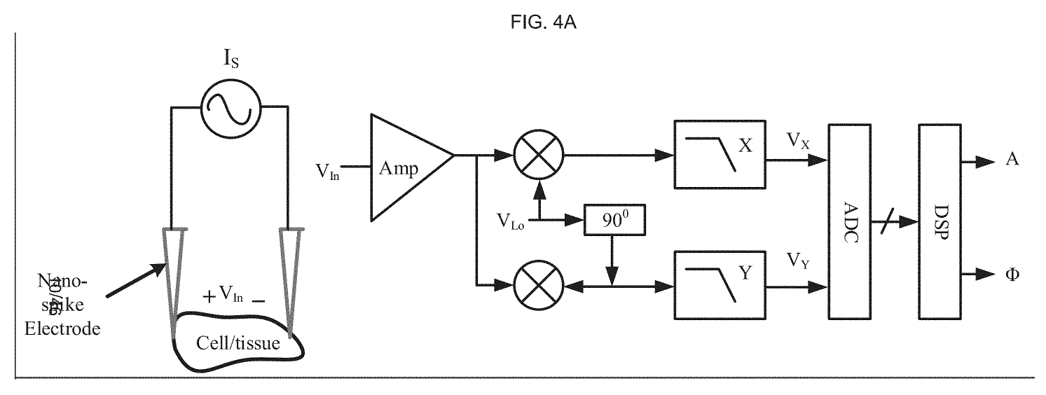

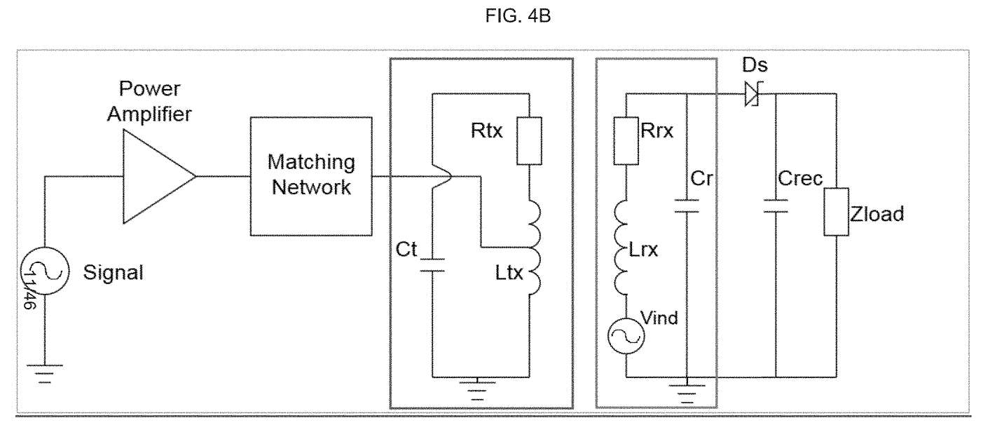

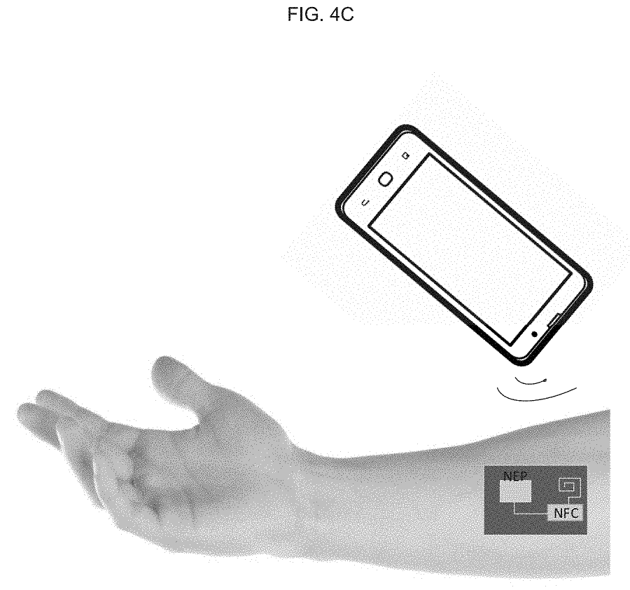

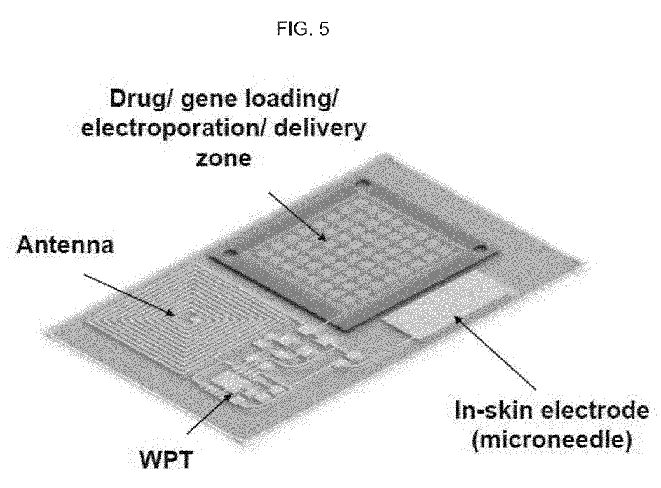

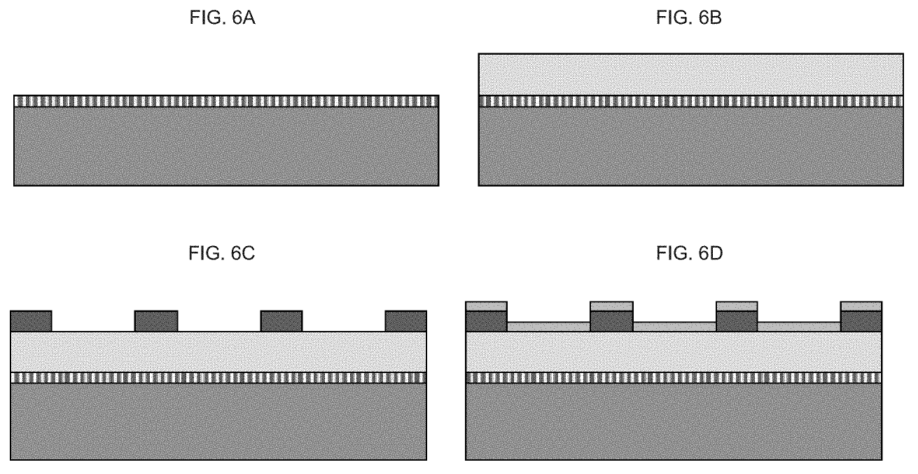

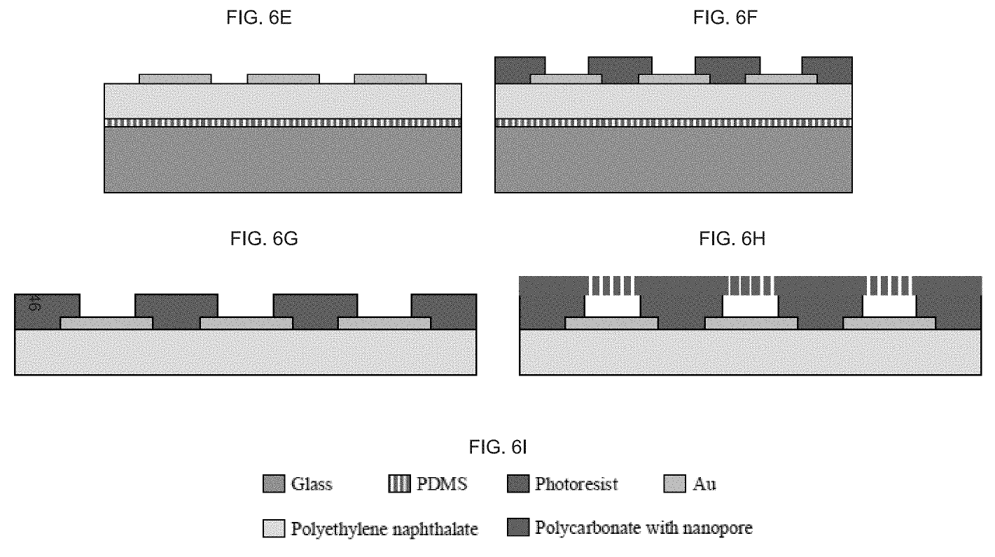

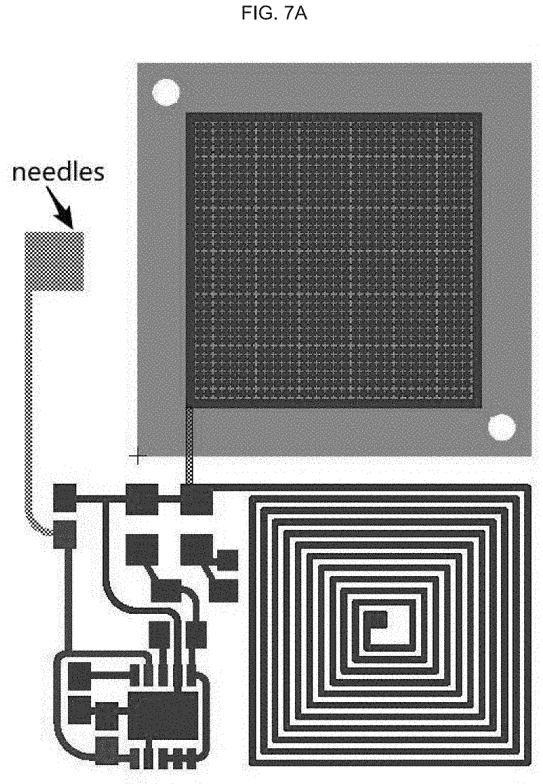

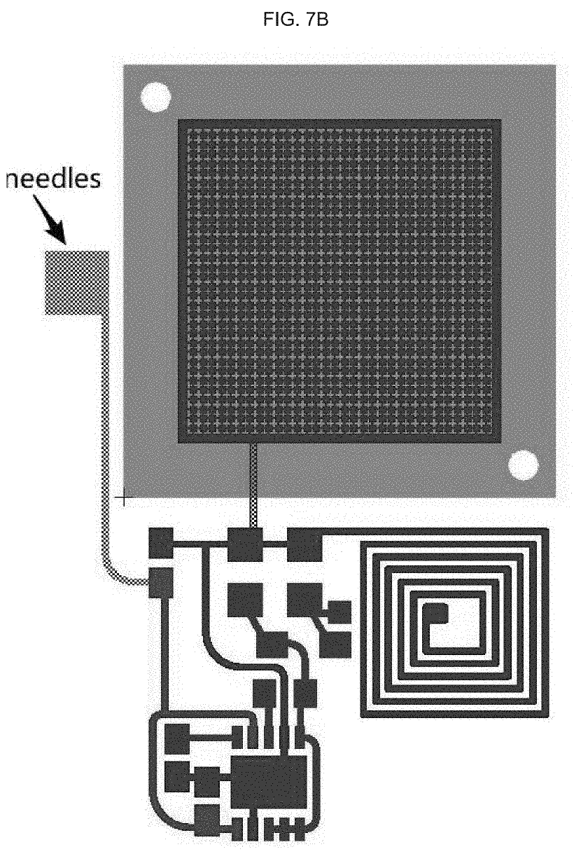

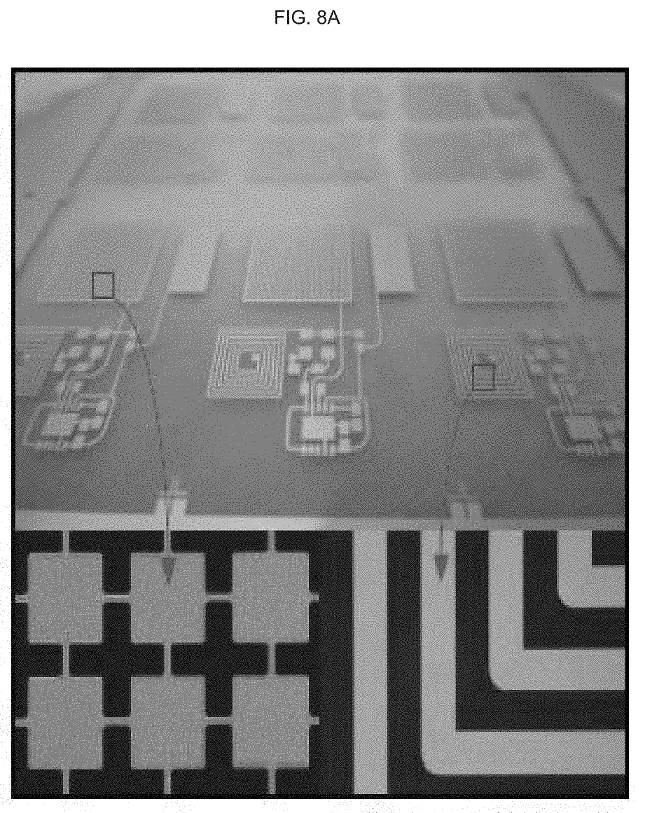

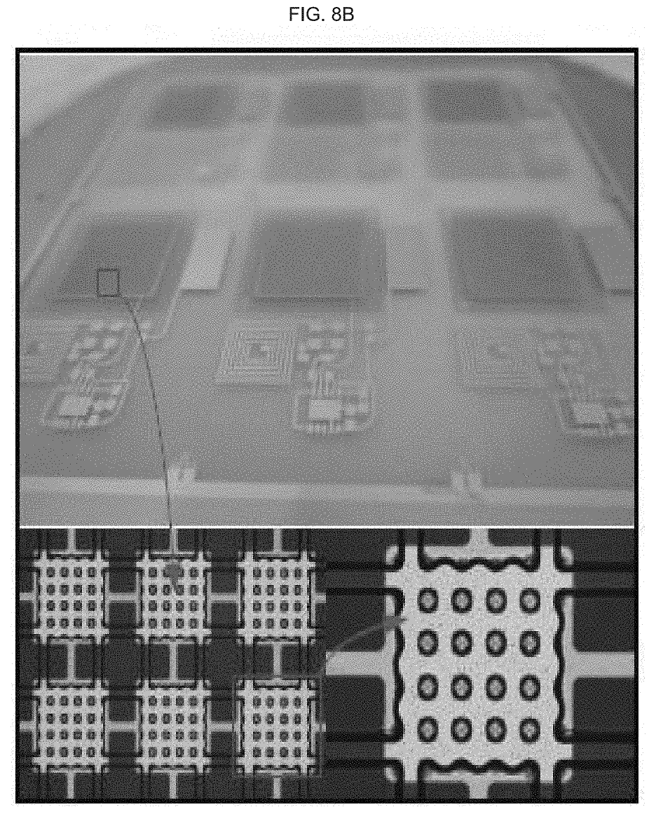

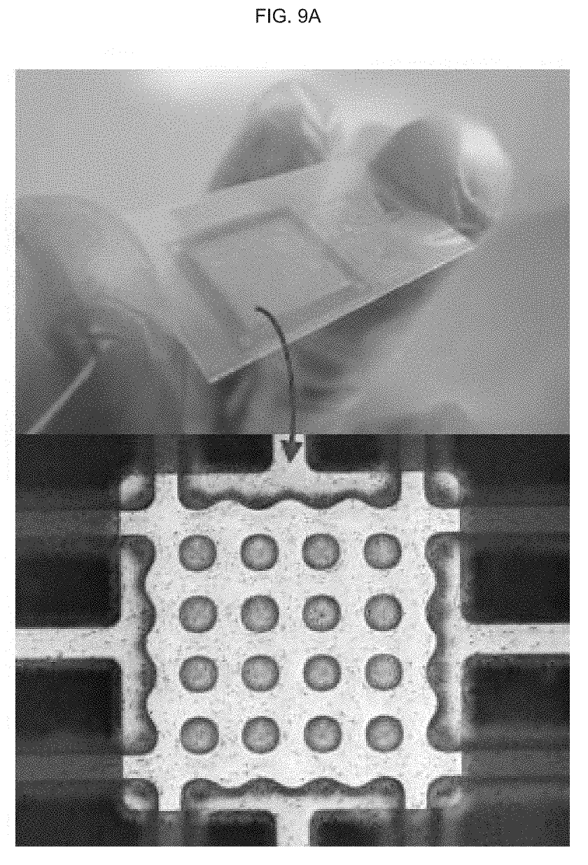

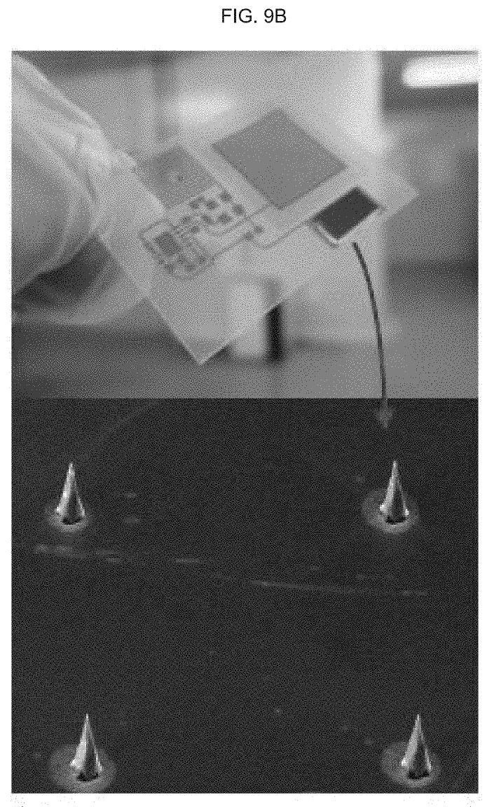

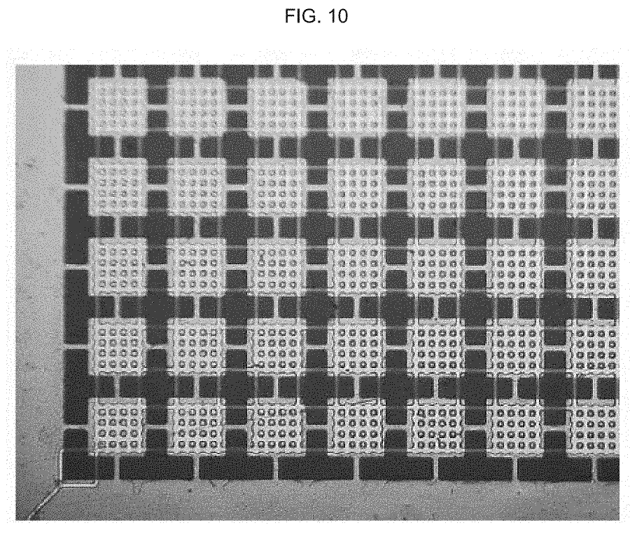

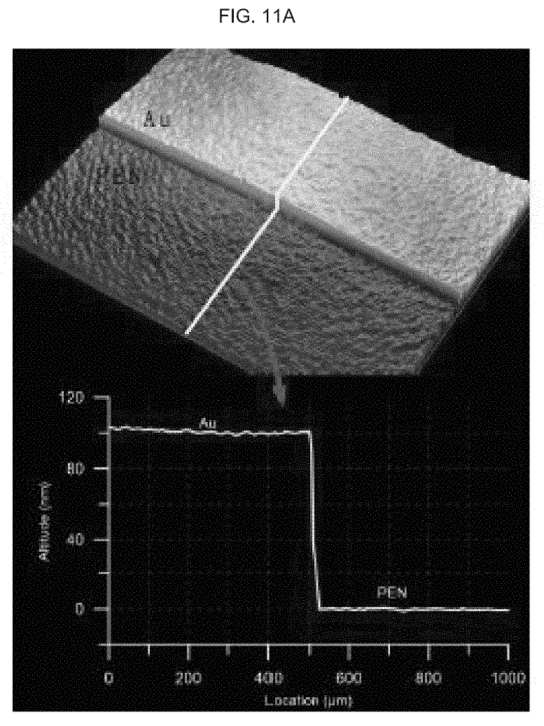

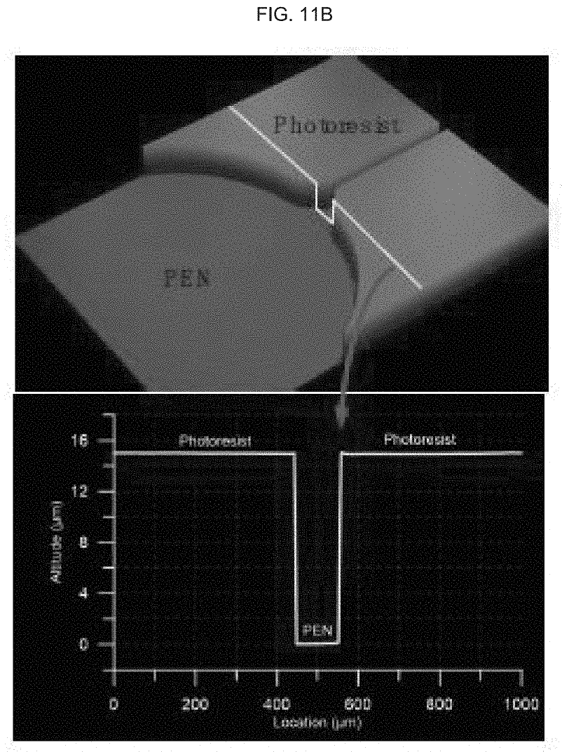

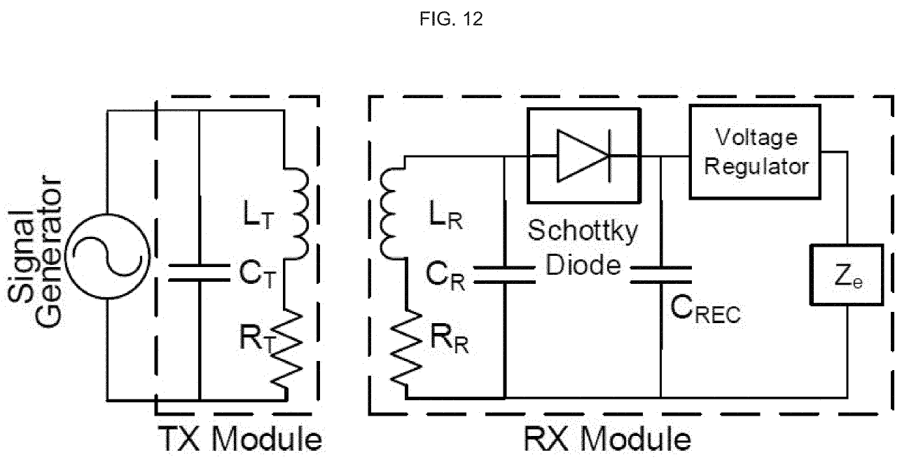

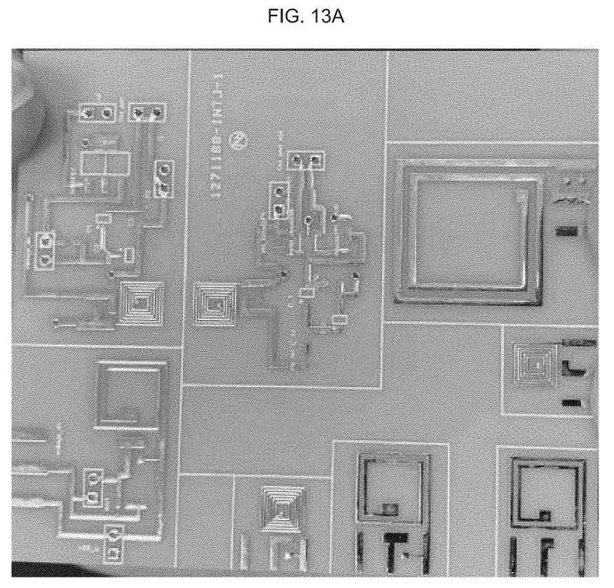

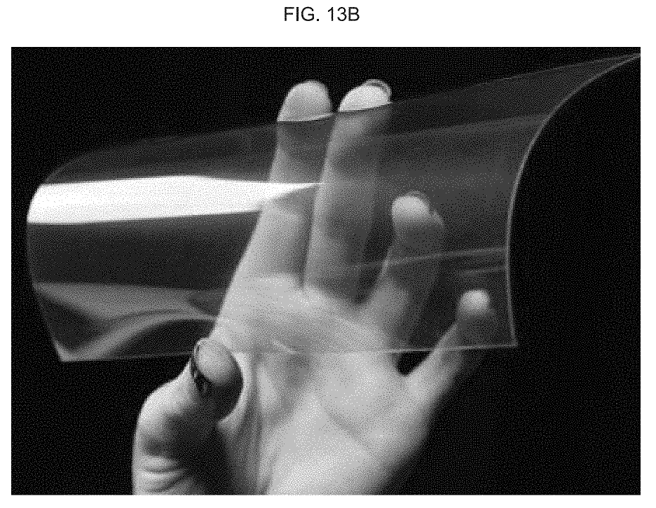

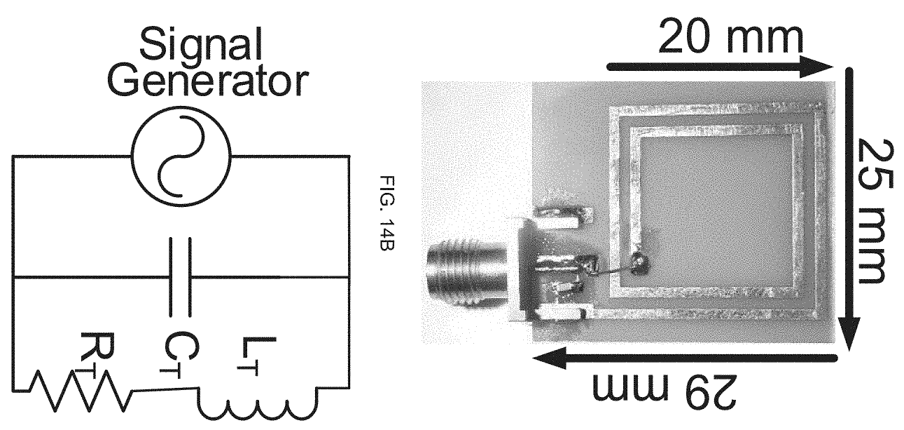

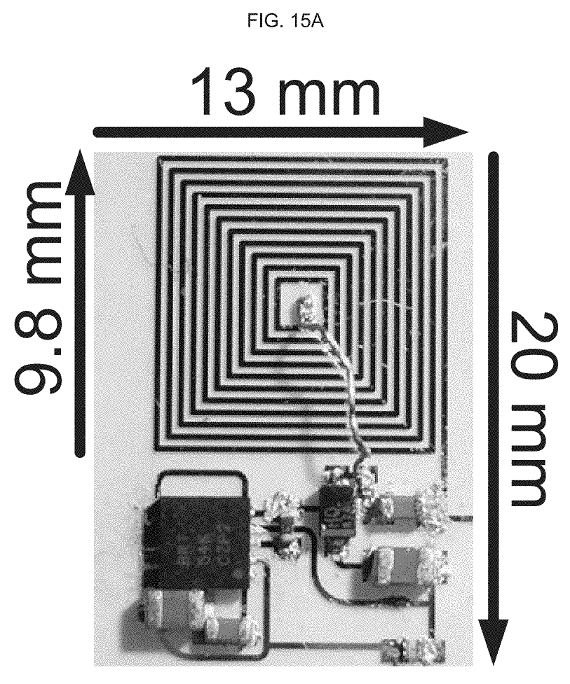

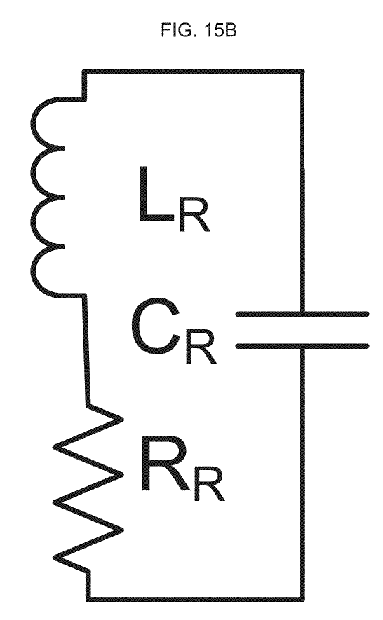

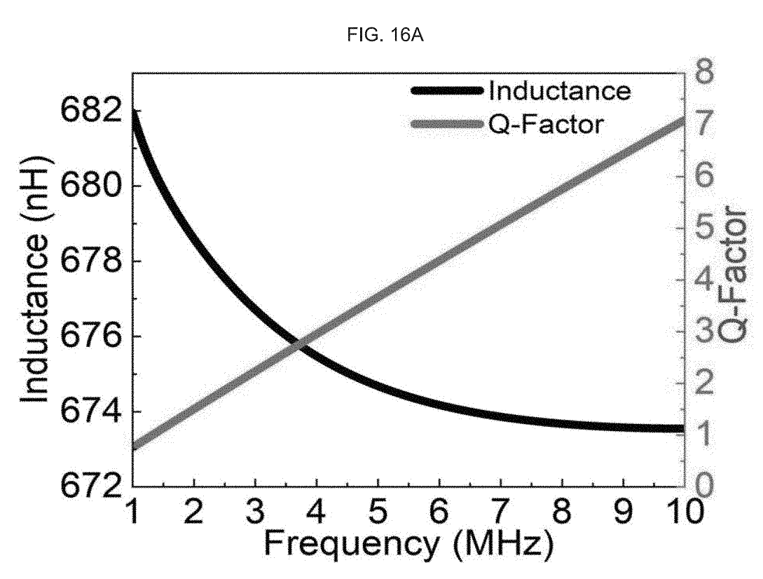

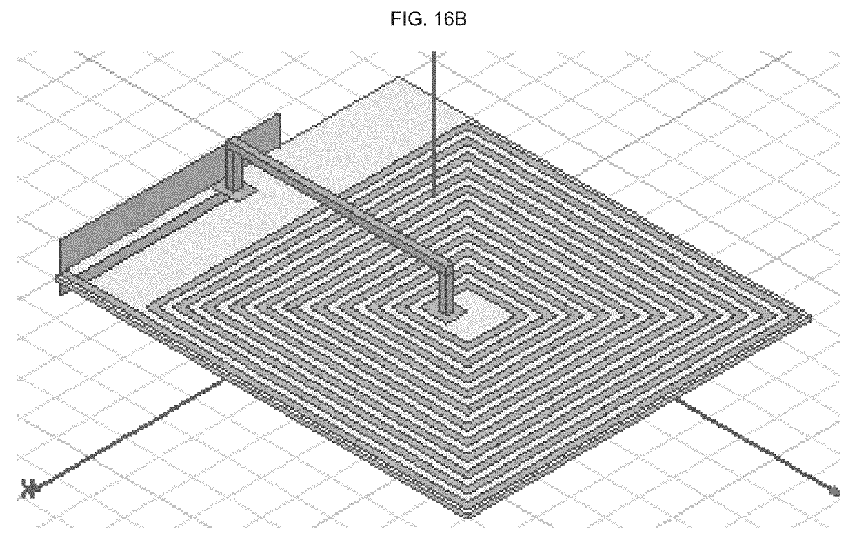

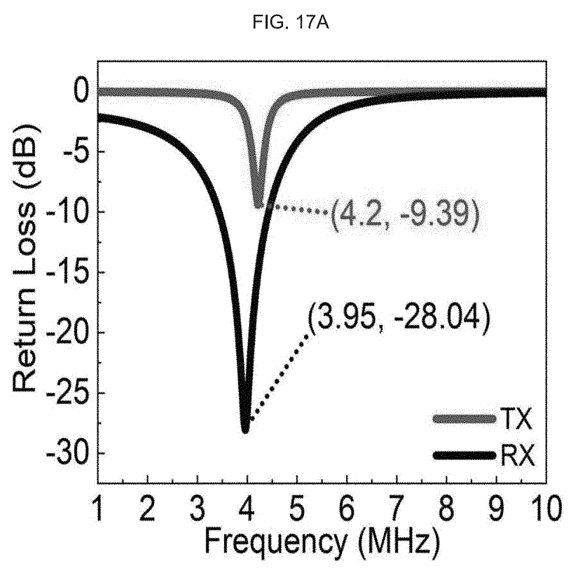

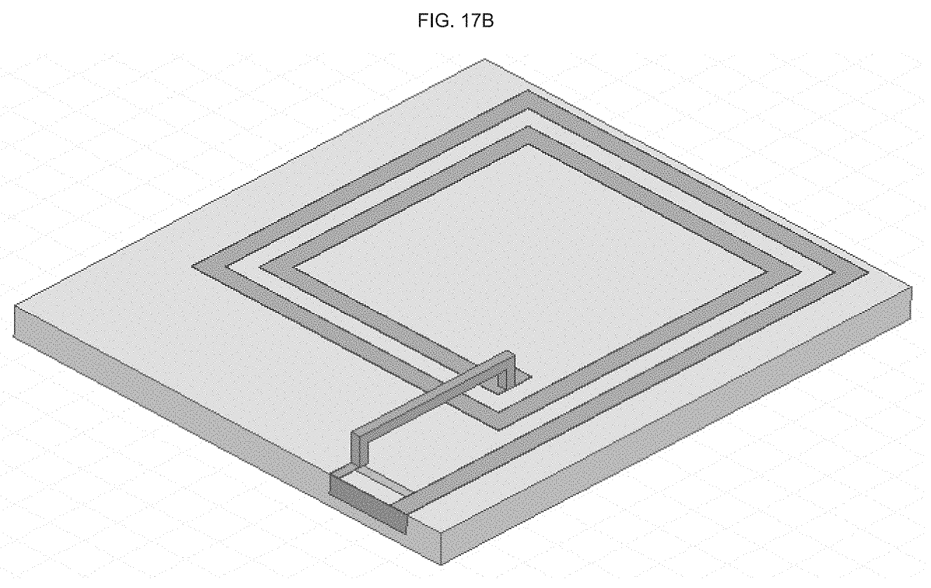

The product of manufacture also comprises a needle, or a so-called “nano-spike electrode” as illustrated in FIG. 4A, which on one end is directly connected (and is operatively connected) to the NFC chip, where the NFC chip is operatively connected to a micro-electrode under the micro-channels; the needle (or “nano-spike electrode”) acts as an electrode connection to the skin or mucosa. The needle (nano-spike electrode) is capable of being inserted/placed in/on the skin or mucosal tissue to act as a “bottom” electrode with the micro-electrode under the micro-channels, which is the “top” electrode. The portion of the needle inserted into or in contact with (or in substantial contact with) the skin or mucosa acts as the corresponding “bottom” micro-electrode array, i.e., as a bottom electrode in contact with the skin or mucosa. In operation, the needle can be directly inserted into or it can be in contact with the skin or mucosa. In summary, the NFC chip is operatively connected to the “top” micro-electrode under the micro-channels such that an electric field from the chip passes to the skin or mucosa, i.e., an electric field from the chip to the skin or mucosa is established, i.e., an electrical connection is made. Circuit is completed via the needle (or “nano-spike electrode”) acting as an electrode connection to the skin or mucosa.

The Macromolecule (or Gene) Delivery zone of the device can concentrate the electric field onto or into the surface of the skin or mucosa; and, because of the electric field, wherein each microchannel in contact with the skin or mucosa creates a small opening (e.g., less than about 1 micron) on or through a skin or mucosa cell's membrane, thereby electrophoretically driving cargo (e.g., macromolecules such as small molecules or nucleic acids) into the cell (e.g., at high speed), i.e., electroporating the cargo into the cell.

The three panels of FIG. 3A show increasingly magnified images of an exemplary Macromolecule (or Gene) Delivery zone (GD): Top Panel: Devices can be batch-produced from one big polymer sheet. After production, each device with three zones (GD, SS, WP) are diced from the polymer sheet for assembly; Lower Left Panel: zoom-in micrograph shows the features of microchannel connection on a single device; Lower Right Panel: Further zoom-in shows one square region of microchannel; and the circle arrays are pillars to support the top nanopore membrane and to define the electric field focusing on nanochannel.

FIG. 3B illustrates how a 99% potential drop is distributed over the nanopore for macromolecular (e.g., gene) electrophoresis, which is only 1% over cell for safe electroporation: the image is by finite element analysis to numerically illustrate the potential drop cross the cell, nanochannel and microchannel; this illustrated simulation demonstrates the high resistivity of the nanochannel, where 90% of potential drop is applied over the nanochannel, and only a small portion on the cell, which offers safe electroporation while creating a strong potential drop over the nanochannel for electrophoresis.

FIG. 3C illustrates a cross-sectional schematic of an exemplary device patched on the skin with zoom-in resolution into cell level. From bottom to up, there are microelectrode array, microchannel, nanopore, cells, interstitial extracellular environment in skin, nano-spike electrode. The pulsed electrode field is applied between a top electrode (nano-spike electrode) and a bottom electrode (micro-electrode array). Cells are tightly connected to the nanopore. The potential drop is analyzed and results are shown in FIGS. 3B and 3C.

FIG. 3D illustrates images of an exemplary mounting nanopore membrane (polycarbonate track etched membrane) on a microchannel, the nanopore membrane forming an insulated microfluidic channel for flow in and flow out the macromolecule solutions.

In alternative embodiments, power for cell electroporation at the GD zone is supplied by the WP zone, which is made up of an ultra-thin magnetic spiral antenna and the near-field communication (NFC) chip. The spiral antenna can be made by gold (Au) (e.g., having a thickness of about 20 nm) deposition in photolithographic patterning. GD and WP can be connected via wire bonding. In alternative embodiments, NFC technology is applied here to wirelessly control the conditions of electroporation by a remote cell phone.

In alternative embodiments, the skin sensor in SS zone is also made with gold layer (e.g., having a thickness of about 20 nm) deposition following photolithography. The sensor can be designed to measure connectivity between the device and the skin. The signal of impedance is collected and transmitted remotely to the remote cell phone via NFC as well.

FIG. 4A-C illustrate: An exemplary on-chip signal generator with a frequency ranging from 1 kHz to 10 MHz is used to perform the impedance spectroscopy. The real and imaginary values of the tissue impedance is transmitted wirelessly via a second NFC chip:

FIG. 4A illustrates an exemplary system level block diagram of the proposed impedance spectroscopy sensor. In alternative embodiments, all the signal processing blocks such as amplifier, mixer, phase-shifter, low-pass filter, analog-to-digital converter and the digital signal processing etc. are fabricated using standard commercially available 130nm CMOS process.

FIG. 4B illustrates a schematic of an exemplary NFC system that wirelessly power the NEP device and optionally also has a separate data communication link. In alternative embodiments, the frequency for the wireless power transfer (WPT) scheme is 13.56 MHz which is different than the frequency for the wireless data telemetry which is 3 MHz. This exemplary WPT module consists of the receiver coil (printed on the polydimethylsiloxane (PDMS) substrate using Au), an on-chip capacitor to make the LC tank resonate at 13.56 MHz, the diode-based rectifier and the capacitor to reduce the ripple at the DC voltage output. In alternative embodiments, apart from the receiver coil, every other component in the WPT module can be implemented using standard commercially available 130 nm CMOS process. In alternative embodiments, the total dimension of the CMOS bare die is 1.5 mm by 1.5 mm and the die is packaged using 3mm by 3 mm Quad Flat No-lead (QFN) package.

FIG. 4C illustrates an exemplary wireless telemetry system between the cell-phone and the nano-device patched in the skin.

In alternative embodiments, as illustrated in FIG. 14, an exemplary wireless power transfer (WPT) module has an operation frequency of about 4 MHz (MegaHertZ). An exemplary transmitting (TX) module is a rigid TX module, having a 20×25 mm² planar spiral with 2 turns, and a copper clad FR4 substrate, with copper traces of about 1 mm wide (see FIG. 14A) (FR-4 (or FR4) is a National Electrical Manufacturers Association (NEMA) grade designation for glass-reinforced epoxy laminate material; FR-4 is a composite material composed of woven fiberglass cloth with an epoxy resin binder that is flame resistant, or self-extinguishing). An exemplary transmitting (TX) module also can comprise a signal generator, e.g., an N5171B EXB™ (Keysight Technologies, Santa Rosa, Calif.) RF (radiofrequency) analog signal generator; and inductance L_{T} can be 38 nH, the resonating capacitor C_{T} can be 11 nF, the resistance R_{T} can be 0.2 ohm Ω, and the quality factor Q can be 37, where:

$$Q = \frac{ω L}{R} L = \frac{{X}_{L}}{2 π f}$$

An exemplary receiving (RX) module is made of material comprising a polyethylene naphthalate (PEN) substrate, with gold traces of e.g., about 100 nm thickness and 0.2 mm wide, and can comprise a 9.8×9.8 mm² planar spiral with ten turns, see e.g., FIG. 14A. Patterns can be fabricated using cleanroom nanofabrication processes. An exemplary receiving (RX) module can have an inductance L_{R} of 676 nH, a resonating capacitor C_{R} of 2200 pF, resistance R_{R} can be 6 ohm Ω. An exemplary receiving (RX) module can comprise a Schottky diode, e.g., a CUS08F30™ model by Toshiba Semiconductor; see e.g., FIG. 15A-B. An exemplary receiving (RX) module can comprise a smoothing capacitor. An exemplary receiving (RX) module can comprise a voltage regulator, e.g., a TPS6120DRCT™ model by Texas Instruments. An exemplary receiving (RX) module can have a minimum voltage input of about 0.3 V, and can have a 1,625 Ω load resistor based on a power requirement of 5 mW:

$$L = \frac{{X}_{L}}{2 π f} Q = \frac{ω L}{R}$$

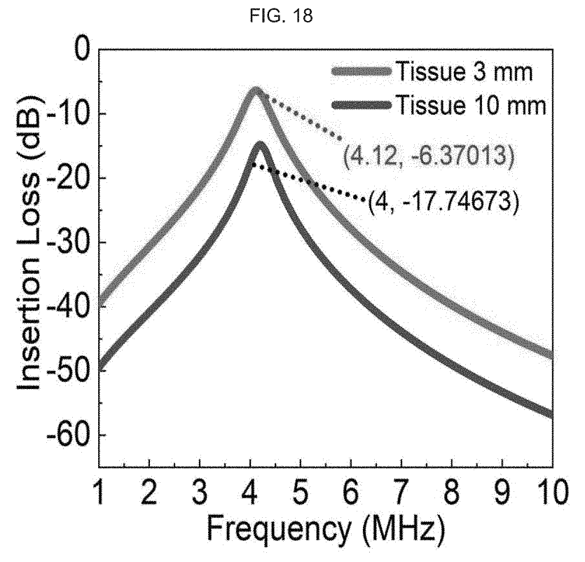

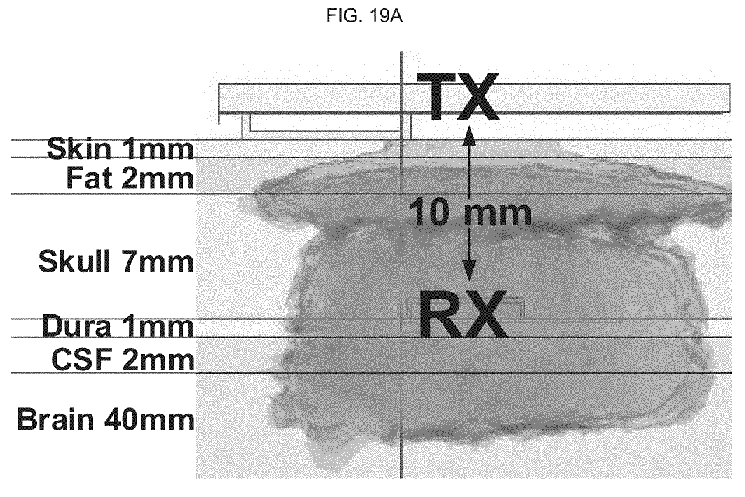

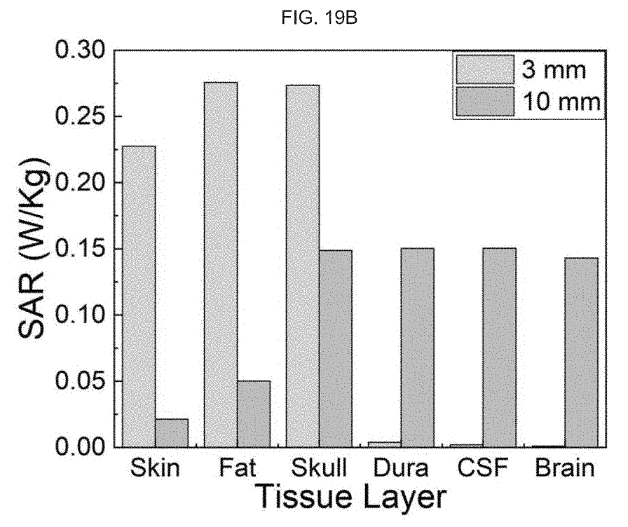

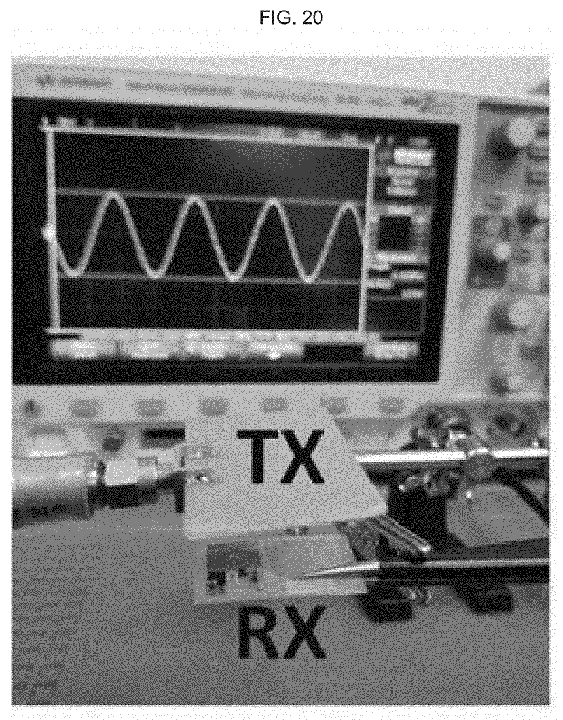

Regarding the studies described in FIGS. 18 to 21, by implementing a wireless power transfer (WPT) system for brain implants, for a 20×13 mm² RX module on a flexible PEN substrate and a 25×29 mm² TX module on rigid FR4 substrate: Measured RX output voltage was: 230.2 mVₚₚ at 5 mm and 168.8 mVₚₚ at 10 mm distance; and Measured Power Transfer Efficiency (PTE) is 7.6% at 5 mm and 4.1% at 10 mm distance.

From the data presented herein, it can be concluded: that the novel skin nanodevices as provided herein can deliver a macromolecular gene (e.g., a NF CRISPR-Cas9) into cells, e.g., cancer cells, e.g., a melanoma cell, with significantly higher efficiency than known commercial systems, e.g., by BioRad; and that novel skin nanodevices as provided herein can deliver FDA approved chemo drugs, including e.g., darcabizine, cisplatin, temozolomide and the like into cells, e.g., cancer cells, e.g., melanoma cells, with significantly higher efficiency than commercial electroporation, direct drug exposure, and chemical methods lipofectamine; and, that novel skin nanodevices as provided herein can precisely control dose of chemo drugs delivered into the cells by tuning the electroporation conditions.

### Kits

Also provided are kits comprising products of manufacture of the invention with instructions for use, and optionally also comprising software for downloading or loading onto a phone, a computer or an equivalent device for allowing or enabling interaction between a user and the product of manufacture, and data transmission from the product of manufacture to the phone, computer or equivalent device, and presentation of the transmitted data to the user, wherein optionally the software is packaged as an app for the phone or equivalent device.

In alternative embodiments, an exemplary “app” (application) will be designed with the Graphical User Interface (GUI) to see the impedance values of the skin area of interest. The data will be transmitted via the same radiating coil that is used for the wireless power transfer application. A switch will be used to switch between the two NFC chips (One is for wireless transmitter/receiver and the other one is for wireless power transfer) periodically. A crystal clock with a frequency of 1 kHz will be used to switch between these two modules. By using the same magnetic spiral antenna for wireless power transfer and wireless telemetry, we reduce the footprint of the sensor substantially.

### Detection

In alternative embodiments, provided are products of manufacture and kits, and methods, for detecting macromolecules, including nucleic acids such as DNA and RNA, including genes and protein-encoding nucleic acids, in skin, epidermal or mucosal cells. In alternative embodiments, products of manufacture and kits, and methods as provided herein are used for delivering genes and macromolecules into cells. Exemplary applications include: 1. Detection; for example, deliver molecular beacon into cells to detect mutation of specific gene implying cancer; and/or 2. Nucleic acid/Gene therapy, deliver transcriptional factor plasmids, gene editing plasmids (e.g. CRISPR-Cas9), miRNAs, into cells for genetic therapy, e.g., therapy for wound cells, cancer cells, cells with genetic conditions, cells effected by an autoimmune disease, and the like.

The invention will be further described with reference to the examples described herein; however, it is to be understood that the invention is not limited to such examples.

### EXAMPLES

### Example 1

### Fabrication of an Exemplary Device

This example describes manufacture of an exemplary device as provided herein.

In alternative embodiment, a method for making an exemplary device of the invention comprises:

(a) coating a substrate comprising a polymeric organosilicon compound, optionally a silicone, a polydimethylsiloxane (PDMS) or an equivalent, on a glass or equivalent, wherein the substrate as an adhesive layer for a polyimide or equivalent;

(b) bonding the polyimide or equivalent to the polymeric organosilicon compound or equivalent using a vacuum followed by a heat treatment,

wherein optionally the vacuum is below about 1 kPa, the temperature is between about 60° C. to 80° C., and/or the bonding time is between about 1 hour (h) to about 3 h;

(c) sputtering chromium and gold on the surface of the photoresist and polyimide or equivalent (a Cr/Au sputtered layer);

wherein optionally the chromium and gold is sputtered on the surface of the photoresist and polyimide or equivalent using magnetron sputtering equipment, and/or the Cr layer is between about 20 nm to 50 nm, and/or the Au layer is between about 200 nm to 500 nm;

(d) patterning a positive photoresist composition on the polyimide or equivalent as a sacrifice layer,

wherein optionally the positive photoresist comprises EPI 680™ (Everlight Chemical, Taiwan), the thickness of patterned photoresist is between about 2 μm to 4 μm, and/or the pattern shape is determined by the Cr/Au layer in step (c), or the pattern has a complementary relationship with Cr/Au structure;

(e) lifting the photoresist, optionally by soaking in an acetone or equivalent solution, optionally for between about 10 min to about 30 min to remove the photoresist completely;

(f) patterning a plurality of micro-channels on the product of manufacture to form a payload delivery zone,

wherein optionally a positive reflowable photoresist AZ P4620™ (Microchemicals GmbH, Ulm, Germany) was used to fabricate a master microchannel mold (optionally as described in Huang, et al. Biomed Microdevices (2012) 14: 873),

wherein the each of the plurality of micro-channels extend to (or protrude from, or almost or substantially extend to) the surface of the product of manufacture such that when all or a section of the product of manufacture is placed on the skin or mucosa each of the plurality of micro-channels on the section of the product of manufacture on the skin makes contact with the skin;

(g) applying an ultra-thin magnetic spiral antenna and a near-field communication (NFC) chip into or onto a section of the product of manufacture, wherein the ultra-thin magnetic spiral antenna is operatively connected to the near-field communication (NFC) chip, wherein the ultra-thin magnetic spiral antenna can receive a remote electromagnetic signal and transmit the signal to a near-field communication (NFC) chip, and the NFC chip is operatively connected to the plurality of micro-channels to further transmit the signal and result in the plurality of micro-channels discharging their aqueous contents out of the plurality of micro-channels when the signal is transmitted;

(g) stripping the polyimide from the glass, wherein optionally the polyimide can be stripped directly by hand.

In one embodiment, an exemplary magnetic spiral antenna is made of a thin layer of gold, which is simultaneously patterned on the polymer substrate with GD microelectrode. NFC chip is mounted on the chip by glue and wire bonded to the antenna and the lead of both microelectrode and impedance sensors.

In one embodiment, macromolecules are injected into the inlet of the microchannel region and will flow through all region to an outlet. The microchannel is an insulated environment after mounting the nanopore membrane on the microchannel.

A number of embodiments of the invention have been described. Nevertheless, it can be understood that various modifications may be made without departing from the spirit and scope of the invention. Accordingly, other embodiments are within the scope of the following claims.

What is claimed is:

1. A product of manufacture for transferring macromolecules into a skin or a mucosal cell, comprising a polyimide or equivalent having embedded thereon or therein:
(a) a plurality of micro-channels forming a payload delivery zone, wherein each of the plurality of micro-channels is capable of holding or storing an aqueous solution,
wherein the each of the plurality of micro-channels extend to (or protrude from, or almost or substantially extend to) the surface of the product of manufacture, or can protrude from the surface, such that when all or a section of the product of manufacture is placed on the skin or mucosa each of or substantially most of the plurality of micro-channels on the section of the product of manufacture makes complete or near contact with the skin or mucosa;
(b) an ultra-thin magnetic spiral antenna and a near-field communication (NFC) chip into or onto a section of the product of manufacture,
wherein the ultra-thin magnetic spiral antenna is operatively connected to the near-field communication (NFC) chip, wherein the ultra-thin magnetic spiral antenna can receive a remote electromagnetic signal and transmit the electromagnetic signal to the NFC chip, and the NFC chip is operatively connected to the plurality of micro-channels to further transmit the electromagnetic signal, or to generate a new or different signal, resulting in the plurality of micro-channels to discharge at least some or substantially all of their aqueous contents out of the plurality of micro-channels when the signal is transmitted; and
(c) a needle or nano-spike electrode capable of being inserted/placed in/on the skin or mucosa, wherein the needle on one end is directly connected (and is operatively connected) to the NFC chip, the NFC chip operatively connected to a micro-electrode under the micro-channels; and the needle or nano-spike electrode acts as an electrode connection to the skin or mucosa acting as a “bottom electrode”, and the NFC chip and micro-electrode under the micro-channels act as a “top” electrode capable of forming an electrode connection to the skin or mucosa when the plurality of micro-channels are in contact with or placed on the skin or mucosal tissue, and the needle or nano-spike electrode when inserted into or in contact with the skin or mucosa acts as the corresponding bottom electrode to complete the circuit,
wherein an electric field is transmitted onto or into the surface of the skin or mucosa each microchannel in contact with the skin or mucosa creates a small opening (optionally less than about 1 micron) on or through a cell's membrane, thereby electrophoretically driving a cargo from within the micro-channels (optionally macromolecules such as small molecules or nucleic acids) into the cell, thereby electroporating the cargo into the cell.

2. The product of manufacture of claim 1, wherein the polyimide or equivalent comprises or is a thermoplastic polyimide, or comprises or is a polyimide comprising: APICAL™; an a poly-oxydiphenylene-pyromellitimide, or KAPTON™; a biphenyl tetracarboxylic dianhydride (BPDA) polymer, or UPILEX™; VTEC PI™; NORTON TH™; KAPTREX™; or any combination thereof.

3. The product of manufacture of claim 1, further comprising a Skin Sensor (SS) capable of measuring the connectivity between the product of manufacture and the skin or mucosa, wherein data generated by the SS is transmitted back to a receiving device (optionally a computer or a cell phone comprising a corresponding receiving and transmitting device).

4. The product of manufacture of claim 1, manufactured as a flexible, wearable, device.

5. A method for making a product of manufacture of claim 1, comprising:
(a) coating a substrate comprising a polymeric organosilicon compound, optionally a silicone, a polydimethylsiloxane (PDMS), a polyethylene naphthalate (PEN) substrate, or an equivalent, on a glass or equivalent, wherein the substrate acts an adhesive layer for a polyimide or equivalent;
(b) bonding the polyimide or equivalent to the polymeric organosilicon compound, polydimethylsiloxane (PDMS), polyethylene naphthalate (PEN) substrate, or equivalents, on a glass or equivalent, using a vacuum followed by a heat treatment, wherein optionally the vacuum is below about 1 kPa, the temperature is between about 60° C. to 80° C., and/or the bonding time is between about 1 hour (h) to about 3 h;
(c) sputtering chromium and gold on the surface of the photoresist and polyimide or equivalent (a Cr/Au sputtered layer);
wherein optionally the chromium and gold is sputtered on the surface of the photoresist and polyimide or equivalent using magnetron sputtering equipment, and/or the Cr layer is between about 20 nm to 50 nm, and/or the Au layer is between about 200 nm to 500 nm;
(d) patterning a positive photoresist composition on the polyimide or equivalent as a sacrifice layer,
wherein optionally the positive photoresist comprises EPI 680™ (Everlight Chemical, Taiwan), the thickness of patterned photoresist is between about 2 μm to 4 μm, and/or the pattern shape is determined by the Cr/Au layer in step (c), or the pattern has a complementary relationship with Cr/Au structure;
(e) lifting the photoresist, optionally by soaking in an acetone or equivalent solution, optionally for between about 10 min to about 30 min to remove the photoresist completely;
(f) patterning a plurality of micro-channels on the product of manufacture to form a payload delivery zone,
wherein optionally a positive reflowable photoresist AZ P4620™ (Microchemicals GmbH, Ulm, Germany) was used to fabricate a master microchannel mold (optionally as described in Huang, et al. Biomed Microdevices (2012) 14: 873),
wherein the each of the plurality of micro-channels extend to (or protrude from, or almost or substantially extend to) the surface of the product of manufacture such that when all or a section of the product of manufacture is placed on the skin or mucosa each of the plurality of micro-channels on the section of the product of manufacture on the skin makes contact with (or nearly or substantially makes contact with) the skin;
(g) applying an ultra-thin magnetic spiral antenna and a near-field communication (NFC) chip into or onto a section of the product of manufacture, wherein the ultra-thin magnetic spiral antenna is operatively connected to the near-field communication (NFC) chip, wherein the ultra-thin magnetic spiral antenna can receive a remote electromagnetic signal and transmit the signal to a near-field communication (NFC) chip, and the NFC chip is operatively connected to the plurality of micro-channels to further transmit the signal and result in the plurality of micro-channels discharging their aqueous contents out of the plurality of micro-channels when the signal is transmitted;
(g) stripping the polyimide from the glass, wherein optionally the polyimide can be stripped directly by hand.

6. The product of manufacture of claim 4, wherein the polyimide or equivalent comprises or is a thermoplastic polyimide, or comprises or is a polyimide comprising: APICAL™; an a poly-oxydiphenylene-pyromellitimide, or KAPTON™; a biphenyl tetracarboxylic dianhydride (BPDA) polymer, or UPILEX™; VTEC PI™; NORTON TH™; KAPTREX™; or any combination thereof.

7. The product of manufacture of claim 1, wherein the plurality of micro-channels each comprise an aqueous solution, and optionally the aqueous solution comprises a payload, and optionally the payload comprises a macromolecule or a small molecule, and optionally the macromolecule comprises a nucleic acid, and optionally the nucleic acid comprises a DNA or an RNA, optionally a gene or protein-encoding nucleic acid.

8. The product of manufacture of claim 1, further comprising either: (a) directly affixing or attaching a needle or a nano-spike electrode (optionally a gold needle or nano-spike electrode, optionally having a diameter of between about 0.5 mm to about 1 mm) to the product of manufacture; or, (b) indirectly connecting a needle or a nano-spike electrode to the product of manufacture by a wire, wherein the needle or the nano-spike electrode is connected to an NFC chip by a wire bonding.

9. A kit comprising: a product of manufacture of claim 1,
wherein optionally the further comprises software for downloading or loading onto a phone, a computer or an equivalent device for allowing or enabling interaction between a user and the product of manufacture, and data transmission from the product of manufacture to the phone, computer or equivalent device, and presentation of the transmitted data to the user, wherein optionally the software is packaged as an app for the phone or equivalent device.

10. A method for: delivering a payload to skin or mucosal cells or to dermal or epidermal cells; or, transferring macromolecules into a skin or a mucosal cell; the method comprising:
(a) applying a product of manufacture of claim 1 to a skin or a mucosa, or a skin cell or a mucosal cell, wherein the plurality of micro-channels of the product of manufacture make complete, substantial, or near contact with the skin; and
(b) transmitting a sufficient electromagnetic signal to the product of manufacture for reception by the ultra-thin magnetic spiral antenna, which transmits this signal to the near-field communication (NFC) chip, and the NFC chip, which is operatively connected to the plurality of micro-channels, further transmits the signal to result in the plurality of micro-channels to discharge some or substantially all or all of their aqueous contents (the payload) out of the plurality of micro-channels,
and optionally the electromagnetic signal is also sufficient to result in an electroporation of some or substantially all or all of the payload into the skin or mucosal cells.
